(19) United States
(12) Patent Application Publication
(10) Pub. No.: US 2025/0034557 A1
(43) Pub. Date: Jan. 30, 2025

(54) KITS AND METHODS FOR PREPARATION OF NUCLEIC ACID LIBRARIES FOR SEQUENCING

(71) Applicant: seqWell, Inc., Beverly, MA (US)

(72) Inventor: Jack T. LEONARD, South Hamilton, MA (US)

(21) Appl. No.: 18/697,064

(22) PCT Filed: Sep. 29, 2022
(86) PCT No.: PCT/US2022/077273

§ 371 Date: Mar. 29, 2024

Related U.S. Application Data
(60) Provisional application No. 63/249,653, filed on Sep. 29, 2021.

(51) Int. Cl. C12N 15/10; C12Q 1/6806; C12Q 1/686; C12Q 1/6874

(52) U.S. Cl. CPC C12N 15/1096; C12Q 1/6806; C12Q 1/686; C12Q 1/6874

(57) ABSTRACT

The present application relates to kits and methods for preparation of nucleic acid libraries for nucleic acid sequencing. More specifically, the invention pertains to more efficient kits and methods for the preparation of nucleic acid libraries through performing all steps in a single reaction vessel.

### SEQUENCE LISTING

The instant application contains a Sequence Listing which has been submitted electronically in XML format and is hereby incorporated by reference in its entirety. Said XML copy, created on Sep. 27, 2022, is named 51178-011WO2_Sequence_Listing_9_27_22 and is 94,151 bytes in size.

### BACKGROUND OF THE INVENTION

Nucleic acid sequencing may involve the preparation of nucleic acid libraries from one or more nucleic acid samples. Methods of preparing nucleic acid libraries used in next generation sequencing may require both fragmentation of long nucleic acid samples to lengths suitable for the sequencing method used, addition of adapter nucleic acids for DNA sequencing and tagging of each library fragment with one or more short identification (e.g., barcode) sequences for identification and analysis. These methods may include multiple steps, with purification required in-between, which can both increase the preparation time as well as introduce errors in the final sequencing result. Provided here are kits and methods for addressing this problem.

### SUMMARY OF THE INVENTION

In general, the present invention relates to kits and methods for the preparation of nucleic acid libraries, e.g., that are suitable for nucleic acid sequencing via next-generation sequencing (NGS) techniques.

In one aspect, the invention provides a kit. The kit includes a first composition including a DNA polymerase; a second composition including a first synaptic complex including a first transposase and a first adapter oligonucleotide; and a second synaptic complex including a second transposase and a second adapter oligonucleotide; wherein the second composition does not include magnesium ions (e.g., Mg²⁺); and magnesium ions (e.g., Mg²⁺) either in a third composition or in the first composition.

In some embodiments, the first adapter oligonucleotide includes a first universal primer region and a first adapter barcode region; and the second adapter oligonucleotide includes a second universal primer region and a second adapter barcode region.

In one aspect, the invention features a method of generating a library from a nucleic acid sample including a target nucleic acid in a single-pot reaction in a first reaction vessel. The method includes amplifying the nucleic acid sample using the kit described herein to generate sequencing oligonucleotides including a nucleic acid sequence including the first universal primer region, the first adapter barcode region, a homologous sequence of a first nucleic acid fragment, the complement sequence of the second adapter barcode region, and the complement sequence of the second universal primer region; and the complement sequence the nucleic acid sequence, thereby generating the library. A nucleic acid duplex including the first nucleic acid fragment and its complement is generated from the nucleic acid sample by transposase activity.

In some embodiments, the method further includes combining the first composition, the second composition, magnesium ions (e.g., Mg²⁺), and the nucleic acid sample in the first reaction vessel; generating intermediate nucleic acids including nucleic acid sequences of the first universal primer region, the first adapter barcode region, and the homologous sequence of the first nucleic acid fragment; and the second universal primer region, the second adapter barcode region, and the complement sequence of the first nucleic acid fragment, in a transposition reaction between the nucleic acid sample, the first synaptic complex, and the second synaptic complex; and generating the sequencing oligonucleotides in a polymerization reaction involving the intermediate nucleic acids and the DNA polymerase, wherein the polymerization reaction extends the 3′ ends of a nucleic acid duplex including a pair of the intermediate nucleic acids to generate the sequencing oligonucleotides.

In some embodiments, the transposition reaction occurs at a transposition reaction temperature between 25-65° C. (e.g., between 35-65° C., between 40-65° C., between 45-65° C., between 50-65° C., between 55-65° C., between 60-65° C., between 25-60° C., between 25-55° C., between 25-50° C., between 25-45° C., between 25-40° C., between 25-35° C., between 25-30° C., between 40-50° C., or between 53-57° C.; e.g., at about 25° C., at about 30° C., about 35° C., about 40° C., about 45° C., about 50° C., about 53° C., about 54° C., about 55° C., about 56° C., about 57° C., about 60° C., or about 65° C.) and/or the polymerization reaction occurs at a polymerization reaction temperature between 55-95° C. (e.g., between 60-95° C., between 65-95° C., between 70-95° C., between 75-95° C., between 80-95° C., between 85-95° C., between 90-95° C., between 55-90° C., between 55-85° C., between 55-80° C., between 55-75° C., between 55-70° C., between 55-65° C., between 55-60° C., between 65-85° C., between 70-80° C., between 73-77° C.; e.g., at about 55° C., at about 60° C., at about 65° C., at about 70° C., at about 73° C., at about 74° C., at about 75° C., at about 76° C., at about 77° C., at about 80° C., at about 85° C., at about 90° C., at about 95° C.).

In some embodiments, the transposition reaction occurs for a first reaction duration between 1 and 30 minutes (e.g., between 1 and 25 minutes, between 1 and 20 minutes, between 1 and 15 minutes, between 1 and 10 minutes, between 10 and 30 minutes, between 15 and 30 minutes, between 20 and 30 minutes, between 25 and 30 minutes, between 10 and 20 minutes, between 15 and 25 minutes; e.g., about 1 minutes, about 10 minutes, about 13 minutes, about 14 minutes, about 15 minutes, about 16 minutes, about 17 minutes, about 20 minutes, about 25 minutes, or about 30 minutes), and/or the polymerization reaction occurs for a second reaction duration between 1 and 60 minutes (e.g., between 1 and 55 minutes, between 1 and 50 minutes, between 1 and 45 minutes, between 1 and 40 minutes, between 1 and 35 minutes, between 1 and 30 minutes, between 1 and 25 minutes, between 1 and 20 minutes, between 1 and 15 minutes, between 1 and 10 minutes, between 10 and 60 minutes, between 15 and 60 minutes, between 20 and 60 minutes, between 25 and 60 minutes, between 30 and 60 minutes, between 35 and 60 minutes, between 40 and 60 minutes, between 45 and 60 minutes, between 50 and 60 minutes, between 55 and 60 minutes, between 15 and 35 minutes, between 30 and 50 minutes, between 10 and 20 minutes, between 20 and 40 minutes, or between 13 and 17 minutes; e.g., about 1 minute, about 5 minutes, about 10 minutes, about 13 minutes, about 14 minutes, about 15 minutes, about 16 minutes, about 17 minutes, about 20 minutes, about 25 minutes, about 30 minutes, about 35 minutes, about 40 minutes, about 45 minutes, about 50 minutes, about 55 minutes, or about 60 minutes).

In some embodiments, the nucleic acid sample, the first adapter oligonucleotide, the second adapter oligonucleotide, the pair of intermediate nucleic acids, and/or the sequencing oligonucleotides include DNA.

In some embodiments, the nucleic acid sample includes double-stranded DNA (dsDNA).

In some embodiments, the method further includes amplifying the library in the first reaction vessel in a PCR reaction with a first universal primer and a second universal primer, and wherein the first universal primer includes a sequence homologous to the first universal primer region and the second universal primer includes a sequence homologous to the second universal primer region, thereby generating an amplified library.

In some embodiments, the library, the first universal primer, and the second universal primer include DNA.

In some embodiments, in the kits described above, the first adapter oligonucleotide includes a first adapter priming region and the second adapter oligonucleotide includes a second adapter priming region.

In some embodiments, the kit includes a first amplifier oligonucleotide including a first universal primer region and a first amplifier priming region; and a second amplifier oligonucleotide including a second universal primer region and a second amplifier priming region, wherein the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

In some embodiments, the kit includes a first amplifier oligonucleotide including a first universal primer region, a first amplifier barcode region, and a first amplifier priming region; and a second amplifier oligonucleotide including a second universal primer region, a second amplifier barcode region, and a second amplifier priming region, wherein the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

In one aspect, the invention features a method of generating a library from a nucleic acid sample in a single-pot reaction in a first reaction vessel. The method includes amplifying the nucleic acid sample using the kit described herein to generate amplicons including a nucleic acid sequence including the first universal primer region, the first amplifier priming region, a homologous sequence of a first nucleic acid fragment, the complement sequence of the second amplifier priming region, and the complement sequence of the second universal primer region; and the complement sequence thereof, thereby generating the library. A nucleic acid duplex including the first nucleic acid fragment and its complement is generated from the nucleic acid sample by transposase activity.

In one aspect, the invention features a method of generating a library from a nucleic acid sample in a single-pot reaction in a first reaction vessel. The method includes amplifying the nucleic acid sample using the kit described herein to generate amplicons including a nucleic acid sequence including the first universal primer region, the first amplifier barcode region, the first amplifier priming region, a homologous sequence of a first nucleic acid fragment, the complement sequence of the second amplifier priming region, the complement sequence of the second amplifier barcode region, and the complement sequence of the second universal primer region; and the complement sequence thereof, thereby generating the library. A nucleic acid duplex including the first nucleic acid fragment and its complement is generated from the nucleic acid sample by transposase activity.

In some embodiments, the method further includes combining the first composition, the second composition, magnesium ions (e.g., Mg²⁺), the first amplifier oligonucleotide, the second amplifier oligonucleotide, and the nucleic acid sample in the first reaction vessel; generating intermediate nucleic acids including nucleic acid sequences of the first adapter priming region and the homologous sequence of the first nucleic acid fragment; and the second adapter priming region and the complement sequence of the first nucleic acid fragment, in a transposition reaction between the nucleic acid sample, the first synaptic complex, and the second synaptic complex; and generating the amplicons in a PCR reaction with a pair of the intermediate nucleic acids, DNA polymerase, the first amplifier oligonucleotide, and the second amplifier oligonucleotide.

In some embodiments, the transposition reaction occurs at a transposition reaction temperature between 25-65° C. (e.g., between 35-65° C., between 40-65° C., between 45-65° C., between 50-65° C., between 55-65° C., between 60-65° C., between 25-60° C., between 25-55° C., between 25-50° C., between 25-45° C., between 25-40° C., between 25-35° C., between 25-30° C., between 40-50° C., or between 53-57° C.; e.g., at about 25° C., at about 30° C., about 35° C., about 40° C., about 45° C., about 50° C., about 53° C., about 54° C., about 55° C., about 56° C., about 57° C., about 60° C., or about 65° C.).

In some embodiments, the transposition reaction occurs for a first reaction duration between 1 and 30 minutes (e.g., between 1 and 25 minutes, between 1 and 20 minutes, between 1 and 15 minutes, between 1 and 10 minutes, between 1 and 5 minutes, between 5 and 10 minutes, between 5 and 20 minutes, between 5 and 30 minutes, between 10 and 30 minutes, between 15 and 30 minutes, between 20 and 30 minutes, between 25 and 30 minutes, between 10 and 20 minutes, between 15 and 25 minutes; e.g., about 1 minute, about 5 minutes, about 10 minutes, about 13 minutes, about 14 minutes, about 15 minutes, about 16 minutes, about 17 minutes, about 20 minutes, about 25 minutes, or about 30 minutes).

In some embodiments, the PCR reaction includes 1-35 cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 cycles).

In some embodiments, the nucleic acid sample, the first adapter oligonucleotide, the second adapter oligonucleotide, the first amplifier oligonucleotide, the second amplifier oligonucleotide, the intermediate nucleic acids, and/or the amplicons include DNA.

In some embodiments, the nucleic acid sample includes double-stranded DNA (dsDNA).

In one aspect, the invention features a method of generating a library including amplicons from a nucleic acid sample in a single-pot reaction in a first reaction vessel. The method includes combining in the first reaction vessel magnesium ions (e.g., Mg²⁺); a DNA polymerase; a first synaptic complex including a first transposase and a first adapter oligonucleotide including a first adapter priming region; a second synaptic complex including a second transposase and a second adapter oligonucleotide including a second adapter priming region; a first amplifier oligonucleotide including a first universal primer region and a first amplifier priming region; and a second amplifier oligonucleotide including a second universal primer region and a second amplifier priming region, wherein the first adapter priming region is homologous to the first amplifier priming region, and the second adapter priming region is homologous to the second amplifier priming region. The method further includes generating intermediate nucleic acids including nucleic acid sequences of the first adapter priming region and a homologous sequence of a first nucleic acid fragment; and the second adapter priming region and a complement sequence of the first nucleic acid fragment, in a transposition reaction between the nucleic acid sample, the first synaptic complex, and the second synaptic complex, wherein a nucleic acid duplex including the first nucleic acid fragment and its complement is generated from the nucleic acid sample by transposase activity. The method further includes generating the amplicons in a PCR reaction involving a pair of the intermediate nucleic acids, the DNA polymerase, the first amplifier oligonucleotide, and the second amplifier oligonucleotide, wherein the amplicons include a nucleic acid sequence including the first universal primer region, the first amplifier priming region, a homologous sequence of the first nucleic acid fragment, the complement sequence of the second amplifier priming region, and the complement sequence of the second universal primer region; and the complement sequence thereof.

In one aspect, the invention features a method of generating a library including amplicons from a nucleic acid sample in a single-pot reaction in a first reaction vessel. The method includes combining in the first reaction vessel magnesium ions (e.g., Mg²⁺); a DNA polymerase; a first synaptic complex including a first transposase and a first adapter oligonucleotide including a first adapter priming region and a first adapter barcode region; a second synaptic complex including a second transposase and a second adapter oligonucleotide including a second adapter priming region and a second adapter barcode region; a first amplifier oligonucleotide including a first universal primer region and a first amplifier priming region; and a second amplifier oligonucleotide including a second universal primer region and a second amplifier priming region, wherein the first adapter priming region is homologous to the first amplifier priming region, and the second adapter priming region is homologous to the second amplifier priming region. The method further includes generating intermediate nucleic acids including nucleic acid sequences of the first adapter priming region, the first adapter barcode region, and a homologous sequence of a first nucleic acid fragment; and the second adapter priming region, the second adapter barcode region, and a complement sequence of the first nucleic acid fragment, in a transposition reaction between the nucleic acid sample, the first synaptic complex, and the second synaptic complex, wherein a nucleic acid duplex including the first nucleic acid fragment and its complement is generated from the nucleic acid sample by transposase activity. The method further includes generating the amplicons in a PCR reaction involving a pair of the intermediate nucleic acids, the DNA polymerase, the first amplifier oligonucleotide, and the second amplifier oligonucleotide, wherein the amplicons include a nucleic acid sequence including the first universal primer region, the first amplifier priming region, the first adapter barcode sequence, a homologous sequence of the first nucleic acid fragment, the complement sequence of the second adapter barcode sequence, the complement sequence of the second amplifier priming region, and the complement sequence of the second universal primer region; and the complement sequence thereof.

In one aspect, the invention features a method of generating a library including amplicons from a nucleic acid sample in a single-pot reaction in a first reaction vessel. The method includes combining in the first reaction vessel magnesium ions (e.g., Mg²⁺); a DNA polymerase; a first synaptic complex including a first transposase and a first adapter oligonucleotide including a first adapter priming region; a second synaptic complex including a second transposase and a second adapter oligonucleotide including a second adapter priming region; a first amplifier oligonucleotide including a first universal primer region, a first amplifier barcode region, and a first amplifier priming region; and a second amplifier oligonucleotide including a second universal primer region, a second amplifier barcode region, and a second amplifier priming region, wherein the first adapter priming region is homologous to the first amplifier priming region, and the second adapter priming region is homologous to the second amplifier priming region. The method further includes generating intermediate nucleic acids including nucleic acid sequences of the first adapter priming region and a homologous sequence of a first nucleic acid fragment; and the second adapter priming region and a complement sequence of the first nucleic acid fragment, in a transposition reaction between the nucleic acid sample, the first synaptic complex, and the second synaptic complex, wherein a nucleic acid duplex including the first nucleic acid fragment and its complement is generated from the nucleic acid sample by transposase activity. The method further includes generating the amplicons in a PCR reaction involving a pair of the intermediate nucleic acids, the DNA polymerase, the first amplifier oligonucleotide, and the second amplifier oligonucleotide, wherein the amplicons include a nucleic acid sequence including the first universal primer region, the first amplifier barcode region, the first amplifier priming region, a homologous sequence of the first nucleic acid fragment, the complement sequence of the second amplifier priming region, the complement sequence of the second amplifier barcode region, and the complement sequence of the second universal primer region; and the complement sequence thereof.

In some embodiments, the transposition reaction occurs at a transposition reaction temperature between 25-65° C.

In some embodiments, transposition reaction occurs for a first reaction duration between 5 and 30 minutes (e.g., between 5 and 25 minutes, between 5 and 20 minutes, between 5 and 15 minutes, between 5 and 10 minutes, between 10 and 30 minutes, between 15 and 30 minutes, between 20 and 30 minutes, between 25 and 30 minutes, between 10 and 20 minutes, between 15 and 25 minutes; e.g., about 5 minutes, about 10 minutes, about 13 minutes, about 14 minutes, about 15 minutes, about 16 minutes, about 17 minutes, about 20 minutes, about 25 minutes, or about 30 minutes).

In some embodiments, the PCR reaction includes 1-35 cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 cycles).

In some embodiments, the nucleic acid sample, the first adapter oligonucleotide, the second adapter oligonucleotide, the first amplifier oligonucleotide, and the second amplifier oligonucleotide, the pair of intermediate nucleic acids, and the amplicons include DNA.

In some embodiments, the nucleic acid sample includes double-stranded DNA (dsDNA).

In one aspect, the invention features a method of generating a library including sequencing oligonucleotides from a nucleic acid sample in a single-pot reaction in a first reaction vessel. The method includes combining in the first reaction vessel magnesium ions (e.g., Mg²⁺); a DNA polymerase; a first synaptic complex including a first transposase and a first adapter oligonucleotide including a first universal primer region and a first adapter barcode region; and a second synaptic complex including a second transposase and a second adapter oligonucleotide including a second universal primer region and a second adapter barcode region. The method further includes generating intermediate nucleic acids including nucleic acid sequences of the first universal primer region, the first adapter barcode region, and a homologous sequence of a first nucleic acid fragment; and the second universal primer region, the second adapter barcode region, and a complement sequence of the first nucleic acid fragment, in a transposition reaction between the nucleic acid sample, the first synaptic complex, and the second synaptic complex, wherein a nucleic acid duplex including the first nucleic acid fragment and its complement is generated from the nucleic acid sample by transposase activity. The method further includes generating the sequencing oligonucleotides in a polymerization reaction involving a pair of the intermediate nucleic acids and the DNA polymerase, wherein the polymerization reaction extends the 3′ ends of a nucleic acid duplex including the pair of intermediate nucleic acids to generate the sequencing oligonucleotides, wherein the sequencing oligonucleotides include a nucleic acid sequence including the first universal primer region, the first adapter barcode region, the homologous sequence of a first nucleic acid fragment, the complement sequence of the second adapter barcode region, and the complement sequence of the second universal primer region; and (b) the complement sequence thereof.

In some embodiments, transposition reaction occurs at a transposition reaction temperature between 25-65° C. (e.g., between 35-65° C., between 40-65° C., between 45-65° C., between 50-65° C., between 55-65° C., between 60-65° C., between 30-60° C., between 30-55° C., between 30-50° C., between 30-45° C., between 30-40° C., between 30-35° C., between 35-55° C., between 40-50° C., or between 53-57° C.; e.g., at about 30° C., about 35° C., about 40° C., about 45° C., about 50° C., about 53° C., about 54° C., about 55° C., about 56° C., about 57° C., about 60° C., or about 65° C.) and/or the polymerization reaction occurs at a polymerization reaction temperature between 55-95° C. (e.g., between 60-95° C., between 65-95° C., between 70-95° C., between 75-95° C., between 80-95° C., between 85-95° C., between 90-95° C., between 55-90° C., between 55-85° C., between 55-80° C., between 55-75° C., between 55-70° C., between 55-65° C., between 55-60° C., between 65-85° C., between 70-80° C., between 73-77° C.; e.g., at about 55° C., at about 60° C., at about 65° C., at about 70° C., at about 73° C., at about 74° C., at about 75° C., at about 76° C., at about 77° C., at about 80° C., at about 85° C., at about 90° C., at about 95° C.).

In some embodiments, the transposition reaction occurs for a first reaction duration between 1 and 30 minutes and/or the polymerization reaction occurs for a second reaction duration between 1 and 60 minutes (e.g., between 1 and 55 minutes, between 1 and 50 minutes, between 1 and 45 minutes, between 1 and 40 minutes, between 1 and 35 minutes, between 1 and 30 minutes, between 1 and 25 minutes, between 1 and 20 minutes, between 1 and 15 minutes, between 1 and 10 minutes, between 1 and 5 minutes, between 5 and 10 minutes, between 10 and 60 minutes, between 15 and 60 minutes, between 20 and 60 minutes, between 25 and 60 minutes, between 30 and 60 minutes, between 35 and 60 minutes, between 40 and 60 minutes, between 45 and 60 minutes, between 50 and 60 minutes, between 55 and 60 minutes, between 15 and 35 minutes, between 30 and 50 minutes, between 10 and 20 minutes, between 20 and 40 minutes, or between 13 and 17 minutes; e.g., about 1 minute, about 5 minutes, about 10 minutes, about 13 minutes, about 14 minutes, about 15 minutes, about 16 minutes, about 17 minutes, about 20 minutes, about 25 minutes, about 30 minutes, about 35 minutes, about 40 minutes, about 45 minutes, about 50 minutes, about 55 minutes, or about 60 minutes).

In some embodiments, the nucleic acid sample, the first adapter oligonucleotide, the second adapter oligonucleotide, the pair of intermediate nucleic acids, and the sequencing oligonucleotides include DNA.

In some embodiments, the nucleic acid sample includes double-stranded DNA (dsDNA).

### Definitions

The following definitions are provided for specific terms, which are used in the disclosure of the present invention:

The term “about”, as used herein, refers to ±10% of a recited value.

By “adapter” or “adapter oligonucleotide” is meant any nucleic acid used to modify a target nucleic acid to make it suitable for amplification or DNA sequencing. In some instances, an adapter may include a nucleic acid sequence for binding transposase known as the transposase mosaic end (ME) sequence. In some instances, an adapter may include a nucleic acid sequence that is homologous or complementary to a nucleic acid sequence used for DNA sequencing. In some instances, an adapter may include a barcode sequence (e.g., a barcode region). In some instances, an adapter may include a nucleic acid sequence for amplification. In some instances, an adapter may be bound to a solid surface. In some instances, an adapter may be bound to a soluble molecular scaffold.

By “amplify” or “amplification” is meant the act or method of creating copies of a nucleic acid molecule. In some instances, the amplification may be achieved using polymerase chain reaction (PCR) or ligase chain reaction (LCR). In other instances, the amplification may be achieved using more than one round of polymerase chain reaction, e.g., two rounds of polymerase chain reaction. In some instances, PCR may be performed using one or more pairs of sequencing oligonucleotides and/or one or more pairs of barcoding oligonucleotides as primers.

By “barcode” is meant a unique oligonucleotide sequence that may allow the corresponding sample to be identified. In some embodiments, the nucleic acid sequence may be located at a specific position in a longer nucleic acid sequence.

By “complement” or “complementary” sequence is meant the sequence of a first nucleic acid in relation to that of a second nucleic acid, wherein when the first and second nucleic acids are aligned antiparallel (5′ end of the first nucleic acid matched to the 3′ end of the second nucleic acid, and vice versa) to each other, the nucleotide bases at each position in their sequences will have complementary structures following a lock-and-key principle (i.e., A will be paired with U or T and G will be paired with C). Complementary sequences may include mismatches of up to one third of nucleotide bases. For example, two sequences that are nine bases in length may have mismatches of at most 3, at most 2, at most 1, or at most 0 nucleotide bases, and remain complementary to one another.

By “flank” is meant the relative positions of three nucleic acid regions. A first and second nucleic acid region is said to flank a third nucleic acid region if the first and second regions lie immediately upstream and downstream of the third nucleic acid region.

By “homologous” is meant having substantially the same sequence. Homologous sequences may differ by up to one third of nucleotide bases. For example, two sequences that are nine bases in length may differ at most by 3, at most by 2, at most by 1, or at most by 0 nucleotide bases, and remain homologous to one another.

By “hybridization” is meant a process in which two single-stranded nucleic acids bind non-covalently by base pairing to form a stable double-stranded nucleic acid. Hybridization may occur for the entire lengths of the two nucleic acids, or only for a portion or subregion of one or both of the nucleic acids. The resulting double-stranded nucleic acid molecule or region is a “duplex.”

By “index-hopping” is meant the phenomenon in nucleic acid sequencing (e.g., via NGS), wherein incorrectly or unexpectedly paired barcodes are detected in the sequencing reads. Index-hopping may also be referred to as, e.g., index-swapping, index crosstalk, index mis-assignment, or index-switching. In instances where nucleic acid sequencing is multiplexed and nucleic acid libraries prepared from multiple nucleic acid samples are sequenced together, each nucleic acid sample may be assigned a unique pair of barcodes. Index-hopping may lead to mis-assignment of sequencing reads to the nucleic acid samples during analysis.

By “library” is meant a collection of nucleic acids that have been prepared for DNA sequencing, wherein the collection of nucleic acids in the library may have the same or different sequences.

By “nucleic acid” is meant a polymeric molecule of at least two linked nucleotides. The terms include, for example, deoxyribonucleic acid (DNA) and ribonucleic acid (RNA), as well as hybrids and mixtures thereof. A nucleic acid may be single-stranded, double-stranded, or contain a mix of regions or portions of both single-stranded or double-stranded sequences. The nucleotides in a nucleic acid are usually linked by phosphodiester bonds, though “nucleic acid” may also refer to other molecular analogs having other types of chemical bonds or backbones, including, but not limited to, phosphoramide, phosphorothioate, phosphorodithioate, O-methyl phosphoramidate, morpholino, locked nucleic acid (LNA), glycerol nucleic acid (GNA), threose nucleic acid (TNA), and peptide nucleic acid (PNA) linkages or backbones. Nucleic acids may contain any combination of deoxyribonucleotides, ribonucleotides, or non-natural analogs thereof. Examples of nucleic acids include, but are not limited to, a gene, a gene fragment, a genomic gap, an exon, an intron, intergenic DNA (including, without limitation, heterochromatic DNA), messenger RNA (mRNA), transfer RNA, ribosomal RNA, ribozymes, small interfering RNA (siRNA), miRNA, small nucleolar RNA (snoRNA), cDNA, recombinant polynucleotides, branched polynucleotides, plasmids, vectors, isolated DNA of a sequence, isolated RNA of a sequence, nucleic acid probes, and primers.

By “nucleotide” or “nt” is meant any deoxyribonucleotide, ribonucleotide, non-standard nucleotide, modified nucleotide, or nucleotide analog. Nucleotides include adenine, thymine, cytosine, guanine, and uracil. Examples of modified nucleotides include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, and 3-(3-amino-3-N-2-carboxypropyl) uracil.

By “oligonucleotide” is meant a nucleic acid up to 150 nucleotides in length. Oligonucleotides may be synthetic. Oligonucleotides may contain one or more chemical modifications, whether on the 5′ end, the 3′ end, or internally. Examples of chemical modifications include, but are not limited to, addition of functional groups (e.g., biotins, amino modifiers, alkynes, thiol modifiers, or azides), fluorophores (e.g., quantum dots or organic dyes), spacers (e.g., C3 spacer, dSpacer, photo-cleavable spacers), modified bases, or modified backbones.

By “synaptic complex” or “transposase synaptic complex” is meant a protein-nucleic acid complex including one or more transposases and one or more oligonucleotides. In some instances, the one or more oligonucleotides of the synaptic complex are inserted into a nucleic acid sequence of a nucleic acid sample by transposase activity. In some instances, the synaptic complex may include a heterodimer of transposase bound to two or more oligonucleotides. In some instances, the insertion of oligonucleotides into the nucleic acid sequence of the nucleic acid sample is preceded by fragmentation of the nucleic acid at the site of insertion by transposase. In some instances, the transposase may be Tn5 transposase or an engineered transposase variant. In some instances, the oligonucleotides may be adapter sequences. In some instances, the synaptic complex is pre-assembled. In some instances, the synaptic complex may be bound to a solid surface. In some instances, the synaptic complex may be bound to a soluble molecular scaffold.

By “target nucleic acid” is meant any nucleic acid (e.g., RNA or DNA) of interest that is selected for amplification or analysis (e.g., sequencing) using a composition (e.g., sequencing oligonucleotides or barcoding oligonucleotides) or method of the invention. In some instances, RNA may be converted to cDNA prior to being treated with a composition of the invention (e.g., sequencing oligonucleotides or barcoding oligonucleotides).

### BRIEF DESCRIPTION OF THE FIGURES

FIG. 1A shows example schematics of two synaptic complexes, a nucleic acid sample, and a DNA polymerase. The synaptic complexes include full length adapter oligonucleotides suitable for methods of generating nucleic acid libraries with barcoded adapter oligonucleotides, wherein the adapter oligonucleotides are barcoded, and includes universal primer regions, an adapter barcode region, a sequencing primer region (SP1 or SP2), and a transposase mosaic end sequence region (ME). The 5′-phosphorylated complement of the ME is also shown (ME′).

FIG. 1B is a schematic showing the resulting product of a transposition reaction of two synaptic complexes on the nucleic acid sample, wherein two adapter oligonucleotides have been covalently attached to the 5′ ends of nucleic acid duplex that includes a target nucleic acid fragment and its complement, generating a pair of intermediate nucleic acids.

FIG. 1C is a schematic showing polymerization reactions that occur on the two strands of the pair of intermediate nucleic acids, after the transposases have dissociated at the polymerization temperature. The direction of polymerization is indicated by the arrows.

FIG. 1D is a schematic showing library fragments after polymerization, wherein the library fragments include (a) a nucleic acid sequence including a first universal primer region, a first barcode region, a first sequencing primer region, a first double-stranded transposase mosaic end sequence region, and a homologous sequence of a first nucleic acid fragment, a complement sequence of the second double-stranded transposase mosaic end sequence region, a complement sequence of the second sequencing primer region, a complement sequence of the second barcode region, a complement sequence of the second universal primer region; and (b) the complement sequence thereof.

FIG. 2A shows example schematics of two synaptic complexes, a nucleic acid sample, a DNA polymerase, and two amplifier oligonucleotides. The synaptic complex includes an adapter oligonucleotide suitable for methods of generating nucleic acid libraries with barcoded amplifier oligonucleotides, wherein the adapter oligonucleotide includes adapter priming regions. The amplifier oligonucleotide includes a universal primer region, amplifier barcode region and an amplifier priming region. In some instances, the adapter priming region is homologous to a corresponding amplifier priming region.

FIG. 2B is a schematic showing the resulting product of a transposition reaction of two synaptic complexes on the nucleic acid sample, wherein two adapter oligonucleotides have been added to the 5′ ends of nucleic acid duplex that includes a nucleic acid fragment and its complement, generating a pair of intermediate nucleic acids.

FIG. 2C is a schematic showing polymerization reactions that occur on the two strands of the pair of intermediate nucleic acids, after the transposases have dissociated at the polymerization temperature. The direction of polymerization is indicated by the arrows.

FIG. 2D shows example schematics of a nucleic acid product of the above polymerization reaction and two exemplary amplifier oligonucleotides. In some instances, the nucleic acid product of the above polymerization reaction may include a first adapter priming region from a first adapter oligonucleotide and a second adapter priming region from a second adapter oligonucleotide. In some instances, a first amplifier oligonucleotide includes a first amplifier priming region including a homologous sequence of the first adapter priming region and a second amplifier oligonucleotide includes a second amplifier priming region including a homologous sequence of the second adapter priming region.

FIG. 2E is a schematic showing amplified library fragments, wherein the amplicons include (a) a nucleic acid sequence including a first universal primer region, an amplifier barcode region, a first amplifier priming region, a first sequencing primer region, a first transposase mosaic end sequence region, a homologous sequence of a first nucleic acid fragment, a complement sequence of a second transposase mosaic end sequence region, a complement sequence of the second sequencing primer region, a complement sequence of the second amplifier priming region, a complement sequence of the second amplifier barcode region, a complement sequence of the second universal primer region; and (b) the complement sequence thereof.

FIG. 3 is a schematic showing the one-step library prep method of the present invention in which tagging and library amplification commence in a single pot reaction.

FIG. 4 is an agarose gel showing the nucleic acid fragment length distribution of a purified library generated by a one-step library preparation method of the invention.

FIG. 5 is a graph showing the normalized read output generated from plasmid DNA over an eight-fold (8-64 ng) input range.

### DETAILED DESCRIPTION

The invention provides new kits and methods of their use to reduce the complexity and time required for generating nucleic acid libraries from nucleic acid samples for nucleic acid sequencing, while simultaneously improving performance of the nucleic acid libraries. The kits and methods are useful in preparing a nucleic acid library suitable for nucleic acid (e.g., DNA or RNA) sequencing through tagging via transposase, and optionally, amplification via polymerase chain reaction (PCR), in a single experimental step and in a one-pot reaction. This inventive approach reduces the complexity of the nucleic acid library preparation workflow by eliminating the need for purification between each step of traditional library preparation. In conventional library prep methods, synaptic complexes i.e., “transposomes” are used to tag nucleic acid samples in an initial reaction step. Then in a second step, the resultant tagged nucleic acid is purified, and then finally in a third step the tagged library is amplified in a separate PCR. In addition to the simplicity of combining all steps of nucleic acid library preparation into a single step, the methods and the use of the kits provided by the invention can be easily multiplexed (e.g., between 1-384 samples simultaneously, or more), can be prepared quickly (e.g., <1 minute per sample for a 96-plex reaction), and can be readily automated with existing technologies for automated sample preparation. The kits and methods provided by the invention can also be readily adapted for a broad range of research and clinical applications. For example, the one-step library preparation method provided by the invention can be easily modified for preparation of nucleic acid libraries without amplification, depending on the method of sequencing to be used. If PCR-free libraries are desired, synaptic complexes can be prepared with full length adapters inclusive of universal primer and barcode sequences that can be loaded on to the transposase. Rather than using the PCR to amplify the library, a simple polymerase fill-in of the adapter (without cycling) is incorporated. Furthermore, the nucleic acid libraries prepared using the kits and methods of the invention provide superior unique dual index (UDI) performance and deliver high-diversity libraries for sequencing. The kits and methods of the present invention have been found to significantly reduce index hopping compared to standard methodologies that employ combinatorial indexing.

### Kits

The invention provides kits that include a first composition that includes DNA polymerase; a second composition that includes a first synaptic complex including a first transposase and a first adapter oligonucleotide, and a second synaptic complex including a second transposase and a second adapter oligonucleotide; and magnesium ions (e.g., Mg²⁺) either in the first composition or in a third composition, but not in the second composition. Magnesium ions may be present with any suitable counter ion, including, but not limited to, chloride, acetate and sulfate. In some instances, the first adapter oligonucleotide includes a first universal primer region and a first adapter barcode region; and the second adapter oligonucleotide includes a second universal primer region and a second adapter barcode region. In some other instances, the first adapter oligonucleotide includes a first adapter priming region; and the second adapter oligonucleotide includes a second adapter priming region. In some instances, the kit may additionally include a first amplifier oligonucleotide, including a first universal primer region, a first amplifier barcode region, and a first amplifier priming region; and a second amplifier oligonucleotide, including a second universal primer region, a second amplifier barcode region, and a second amplifier priming region. Alternatively, in some instances, the kit may additionally include a first amplifier oligonucleotide, including a first universal primer region and a first amplifier priming region; and a second amplifier oligonucleotide, including a second universal primer region and a second amplifier priming region, i.e., without a first or a second amplifier barcode region. In some instances, the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

It will be understood that each component of the kits described herein can be packaged individually; however, use of fewer total compositions is advantageous for ease of use.

### Compositions

As described above, the kits may include a first composition, a second composition, and optionally a third composition. In some instances, the first composition may include DNA polymerase. In some instances, the DNA polymerase may be thermostable, or functional at elevated temperatures (e.g., between 50-100° C., between 50-97° C., between 50-90° C., between 50-80° C., between 50-70° C., between 50-60° C., between 60-97° C., between 70-97° C., between 80-97° C., between 60-80° C., between 60-90° C., between 70-90° C.). In some instances, the DNA-polymerase may be heat-activated or hot-start DNA polymerase. In some instances, the heat-activated or hot-start DNA polymerase may be bound to a heat-labile adduct, e.g., an antibody or aptamer. In some instances, the amount of DNA polymerase in the first composition may be suitable for use in the methods of the invention, e.g., about 0.1 ng/μl, 0.25 ng/μl, 0.5 ng/μl, 0.75 ng/μl, 1 ng/μl, 1.5 ng/μl, 2 ng/μl, 2.5 ng/μl, 3 ng/μl, 3.5 ng/μl, 4 ng/μl, 4.5 ng/μl, or 5 ng/μl. In some instances, the ratio of DNA polymerase to nucleic acid sample may be about 1:1, 1:2, 1:3, 1:4, 1:5, 1:6, 1:7, 1:8, 1:9, 1:10, 1:20, 1:30, 1:40, 1:50, or 1:100. In some instances, the first composition may include nucleotides, e.g., dNTPs (e.g., dATPs, dCTPs, dGTPs, dTTPs, and/or combinations thereof). In some instances, there is sufficient dNTPs in the first composition for use in the methods of the invention. In some instances, the concentration of each dNTP is about 0.1 mM, 0.2 mM, 0.3 mM, 0.4 mM, 0.5 mM, 0.6 mM, 0.7 mM, 0.8 mM, 0.9 mM, or 1 mM (e.g., between 0.1 mM and 1 mM). In one instance, the concentration of each dNTP is about 0.32 mM (e.g., between 0.1 mM and 0.5 mM or between 0.2 mM and 0.4 mM). In some instances, the first composition may include a buffering agent, e.g., Tris, TAPS (e.g., about 16 mM; e.g., between 1 mM and 30 mM or between 10 mM and 20 mM), HEPES, or suitable equivalents thereof. In some instances, the first composition may be buffered to a pH suitable for DNA polymerase and polymerization reactions, e.g., about 8.5. In some instances, the first composition may include magnesium ions (e.g., Mg²⁺) and a suitable counter ion, including, but not limited to, chloride and sulfate. In some instances, the first composition may include magnesium chloride (MgCl₂). In some instances, the concentration of magnesium chloride is suitable for polymerization reactions. In some instances, magnesium chloride is provided in the first composition at a concentration of about 0.5 mM, 1 mM, 2 mM, 3 mM, 4 mM, 5 mM, 6 mM, 7 mM, 8 mM, 9 mM, or 10 mM (e.g., between 0.5 mM and 10 mM). In one instance, the concentration of magnesium chloride is about 3.2 mM (e.g., between 1 mM and 5 mM). In some instances, the first composition may include additives for lowering GC bias during preparation of the sequencing library. In some instances, the first composition may contain suitable amounts or concentrations of other chemical components that enhance DNA polymerase activity or long-term stability (e.g., over days, weeks, months, years; e.g., between 1 and 31 days; between one and four weeks, between 1 and 12 months, or between 1 and 10 years, or more), including, but not limited to, glycerol, TRITON® X-100, DMSO, betaine, potassium chloride, ammonium sulfate, TMAC, Tween 20, bovine serum albumin, and PEG 8000 (e.g., about 5% w/v; e.g., about 5.12% w/v; e.g., between 1% and 10% w/v or between 3% and 7% w/v).

In some instances, the second composition may include a first synaptic complex, including a first transposase and a first adapter oligonucleotide; and a second synaptic complex, including a second transposase and a second adapter oligonucleotide. In some instances, the first and second transposases are suitable for use in a reaction to fragment a dsDNA molecule and add the first and second adapter oligonucleotides to each of the 5′ ends of the two strands of the dsDNA molecule. In some instances, the first and second transposases may be any transposase enzyme, including a DDE transposase enzyme such as a prokaryotic transposase enzyme (e.g., ISs, Tn3, Tn5, Tn7, and Tn10, bacteriophage transposase enzyme from phage Mu (Nagy and Chandler 2004, reviewed by Craig et al. 2002; U.S. Pat. No. 6,593,113)), eukaryotic “cut and paste” transposase enzymes (Jurka et al. 2005; Yuan and Wessler 2011), and retroviral transposases, such as HIV (Dyda et al. 1994; Haren et al. 1999; Rice et al. 1996; Rice and Baker 2001). In some instances, the first and second transposases are Tn5 transposases. In some instances, the first and second adapter oligonucleotides may additionally include a first and/or second transposon end sequence. The first and/or second transposon sequences may be any transposon sequence (e.g., a transposon end sequence), including prokaryotic transposons (e.g., from prokaryotic sources, such as ISs, Tn3, Tn5, Tn7, and Tn10, and bacteriophage included phage Mu (Nagy and Chandler 2004, reviewed by Craig et al. 2002)), eukaryotic “cut and paste” transposons (Jurka et al. 2005; Yuan and Wessler 2011), or any transposon sequence from retroviruses such as HIV (Dyda et al. 1994; Haren et al. 1999; Rice et al. 1996; Rice and Baker 2001). In some instances, the first and second transposon end sequences are Tn5 transposon end sequences. In some instances, the second composition may include a buffering agent, e.g., Tris, TAPS (e.g., about 20 mM (e.g., between 1 mM and 50 mM, between 10 mM and 30 mM, or between 15 mM and 30 mM), HEPES, or suitable equivalents thereof. In some instances, the second composition may be buffered to a pH suitable for synaptic complexes and transposition reactions, e.g., about 8.5 (e.g., between 7 and 10 or between 8 and 9). In some instances, the second composition may contain other chemical components that enhance transposase activity or long-term stability, including, but not limited to, glycerol (e.g., about 50% v/v (e.g., between 10% and 70% w/v or between 40% and 60% w/v), TRITON® X-100 (e.g., about 1% v/v; e.g., between 0.1% and 5% w/v or between 0.5% and 2% w/v), DMSO, betaine, potassium chloride, sodium chloride (e.g., about 100 mM (e.g., between 10 mM and 300 mM, or between 50 mM and 150 mM), ammonium sulfate, TMAC, Tween 20, bovine serum albumin, dithiothreitol (DTT; e.g., about 1 mM; e.g., between 0.1 mM and 5 mM or between 0.5 mM and 2 mM; e.g., about 0.1 mM, 0.15 mM, 0.2 mM, 0.25 mM, 0.3 mM, 0.4 mM, 0.5 mM, 0.6 mM, 0.7 mM, 0.8 mM, 0.9 mM, 1 mM, 1.25 mM, 1.5 mM, 1.75 mM, or 2 mM) and PEG 8000. In some instances, the second composition includes EDTA. In any of the above instances of the invention, the second composition does not include magnesium. In some instances, the concentration of magnesium in the second composition is substantially zero (e.g., less than 1 mM, less than 1 μM, less than 1 nM, less than 1 pM, less than 1 fM, less than 1 aM, or less) and/or the level of magnesium in the second composition is substantially undetectable. In some instances, transposon end sequences may be 5-30 nucleotides (e.g., 5, 6, 7, 8, 9, 10, 11, 12, 13, 14, 15, 16, 17, 18, 19, 20, 21, 22, 23, 24, 25, 26, 27, 28, 29, or 30 nt) in length. In some instances, transposon end sequences may be 19 nt in length.

In some instances, the kit may optionally include a third composition including magnesium ions (e.g., Mg²⁺) and a suitable counter ion, including, but not limited to, chloride and sulfate. In some instances, the third composition may include magnesium chloride (MgCl₂). In some instances, the concentration of magnesium chloride is suitable for polymerization reactions. In some instances, magnesium chloride is provided in the third composition at a concentration of about 0.5 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 40 mM, 50 mM, or higher, e.g., 1 mM or higher. In some instances, the working concentration of magnesium chloride after combining the first, second, and third compositions is between 0.5 mM and 5 mM or 1 mM and 5 mM (e.g., between 0.5 mM and 4 mM, between 0.5 mM and 3 mM, between 0.5 mM and 2 mM, between 0.5 mM and 1 mM, between 1 mM and 5 mM, between 2 mM and 4 mM, between 2.5 and 5 mM, between 2.5 and 3.5 mM, or between 3 and 3.5 mM; e.g., about 0.5 mM, 0.8 mM, 1 mM, 1.5 mM, 2 mM, 2.5 mM, 3 mM, 3.2 mM, 3.5 mM, 4 mM, 4.5 mM, or 5 mM). In one instance, the working concentration of magnesium chloride after combining the first, second, and third compositions is about 0.8 mM (e.g., between 0.5 and 2 mM or between 0.6 and 1.0 mM).

In any of the above instances, the compositions may be in an aqueous solution. In any of the above instances, the first, second, or optionally third composition may include a first amplifier oligonucleotide, including a first universal primer region, a first amplifier barcode region, and a first amplifier priming region; and a second amplifier oligonucleotide, including a second universal primer region, a second amplifier barcode region, and a second amplifier priming region. Alternatively, in any of the above instances, the first, second, or optionally third composition may include a first amplifier oligonucleotide, including a first universal primer region and a first amplifier priming region; and a second amplifier oligonucleotide, including a second universal primer region and a second amplifier priming region, i.e., without a first or a second amplifier barcode region. In some instances, the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

It will be understood that each component of the compositions described herein can be packaged individually; however, use of fewer compositions is advantageous for ease of use. Compositions may be in solution or in solid form. Solvents (e.g., a buffer or water) may be included and/or used to dissolve solid components and/or adjust concentrations.

### Adapter Oligonucleotides

In some instances, the invention provides compositions that include a first adapter oligonucleotide and second adapter oligonucleotide. In some instances, the first adapter oligonucleotide includes, from 5′ to 3′, a first universal primer region, a first adapter barcode region, a first sequencing primer region and a first transposase mosaic end sequence; and the second adapter oligonucleotide includes, from 5′ to 3′, a second universal primer region, a second adapter barcode region, a second sequencing primer region and a second transposase mosaic end sequence. In other instances, the first adapter oligonucleotide includes a first adapter priming region and a first transposase mosaic end sequence; and the second adapter oligonucleotide includes a second adapter priming region and a second transposase mosaic end sequence. In some instances, the first and second universal primer regions and the first and second adapter priming regions may act as priming regions during PCR. In some instances, the adapter oligonucleotides are attached to a solid surface such as a bead or a sequencing flow cell. After hybridizing a complementary oligonucleotide to the transposase mosaic end sequence on the adapter oligonucleotide, synaptic complexes are prepared by incubating transposase protein with duplexed adapter oligonucleotide in a buffered magnesium-free solution for one hour at room temperature. In some instances, the transposase protein and duplexed adapter oligonucleotide are present at 10-20 μM during synaptic complex formation.

Each region of the first and second adapter oligonucleotides (e.g., each universal primer region, each adapter barcode region, and/or each adapter priming region) may include 5-30 nt (e.g., 5, 6, 7, 8, 9, 10, 11, 12, 13, 14, 15, 16, 17, 18, 19, 20, 21, 22, 23, 24, 25, 26, 27, 28, 29, or 30 nt). The overall sequences of the first and second adapter oligonucleotides are chosen to be non-naturally occurring. In some instances, the adapter oligonucleotides may include RNA, DNA, or a combination thereof. In some instances, the first and second adapter oligonucleotides may also contain modified nucleotides, e.g., modified bases, sugars, or phosphates. In some instances, 1-30 nt spacers (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nt) may be included to separate regions on adapter oligonucleotides for adapter assembly, adapter cleavage or annealing sequencing primers.

### Amplifier Oligonucleotides

In some instances, the invention provides compositions that include a first amplifier oligonucleotide and second amplifier oligonucleotide. In some instances, the first amplifier oligonucleotide includes, from 5′ to 3′, a first universal primer region, a first amplifier barcode region, and a first amplifier priming region; and the second amplifier oligonucleotide includes, from 5′ to 3′, a second universal primer region, a second amplifier barcode region, and a second amplifier priming region. Alternatively, in some instances, the first amplifier oligonucleotide includes, from 5′ to 3′, a first universal primer region and a first amplifier priming region; and the second amplifier oligonucleotide includes, from 5′ to 3′, a second universal primer region and a second amplifier priming region, i.e., without a first or a second amplifier barcode region.

Each region of the first and second amplifier oligonucleotides (e.g., each universal priming region, amplifier barcode region, and/or amplifier priming region) may include 5-30 nt (e.g., 5, 6, 7, 8, 9, 10, 11, 12, 13, 14, 15, 16, 17, 18, 19, 20, 21, 22, 23, 24, 25, 26, 27, 28, 29, or 30 nt). The overall sequences of the first and second amplifier oligonucleotides are chosen to be non-naturally occurring. In some instances, the amplifier oligonucleotides may include RNA, DNA, or a combination thereof. In some instances, the first and second amplifier oligonucleotides may also contain modified nucleotides, e.g., modified bases, sugars, or phosphates. In some instances, phosphorothioate linkages are incorporated into the first and second amplifier oligonucleotides to increase resistance to nuclease activity. In some instances, amplifier oligonucleotides are dissolved in 10 mM Tris-HCl, pH 8.0 or ultrapure water.

In a particular instance, first and second compositions including the following lists of components at or about the listed concentrations and conditions may be suitable for use in a method of constructing a nucleic acid library from a nucleic acid sample.

### First Composition

2 ng/μl Hot Start DNA polymerase
0.32 mM dNTPs (each)
3.2 mM magnesium chloride
16 mM TAPS buffer, pH 8.5
5.12% PEG 8000 (w/v)

### Second Composition

500 nM Synaptic complex 1
500 nM Synaptic complex 2
50% glycerol (vol/vol)
20 mM TAPS buffer, pH 8.5
1% TRITON® X-100 (vol/vol)
0.1 mM EDTA
1 mM dithiothreitol
100 mM sodium chloride
8 μM Amplifier oligonucleotide 1
8 μM Amplifier oligonucleotide 2

### DNA (Nucleic Acid Sample)

12.5 ng/μl of purified human DNA (NA12878, Coriell Labs) in 10 mM Tris-HCl, pH 8.

In one instance, a 32 μl reaction is formulated by adding 4 μl of DNA to 8 μl of Component A, and then adding 20 μl of Component B.

### Methods

The invention features methods to generate nucleic acid libraries suitable for sequencing (e.g., by NGS methods) using the compositions and kits of the invention. In some instances, the generated nucleic acid libraries may include sequencing oligonucleotides. In some instances, the sequencing oligonucleotides may be further amplified in a PCR reaction with a first universal primer and a second universal primer, wherein the first universal primer and the second universal primer bind to respective complementary universal primer regions in the sequencing oligonucleotides. In other instances, the generated nucleic acid libraries may include amplicons. In some instances, the amplicons may be further amplified in a PCR reaction with a first universal primer and a second universal primer, wherein the first universal primer and the second universal primer bind to respective complementary universal primer regions in the amplicons.

Methods for Generating Nucleic Acid Libraries with Barcoded Adapter Oligonucleotides

The invention provides methods for the generation of nucleic acid libraries having sequencing oligonucleotides using barcoded adapter oligonucleotides. In some instances, the methods include generating the nucleic acid libraries using (a) a DNA polymerase; (b) a first synaptic complex including a first transposase and a first adapter oligonucleotide, and a second synaptic complex including a second transposase and a second adapter oligonucleotide; and (c) magnesium ions (e.g., Mg²⁺). In some instances, each adapter oligonucleotide includes a universal primer region and an adapter barcode region.

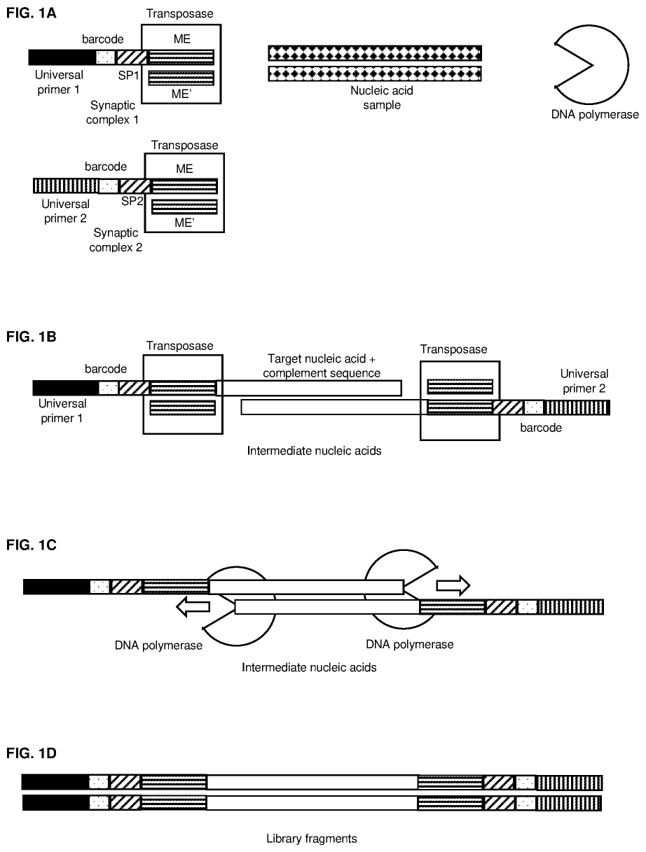

As depicted in FIGS. 1A-1D, the sequencing oligonucleotides may be generated in a single reaction vessel through a method that includes a transposition reaction and a polymerization reaction. As depicted in FIG. 1A, the method includes first combining the DNA polymerase, the first synaptic complex, the second synaptic complex, magnesium ions (e.g., Mg²⁺), and the nucleic acid sample in a first reaction vessel. As depicted in FIG. 1B, the transposases of the synaptic complexes will fragment the nucleic acid sample into nucleic acid fragments, and attach the adapter oligonucleotide sequences to the 5′ ends of the two strands of a nucleic acid duplex containing a nucleic acid fragment and its complement sequence in a transposition reaction to generate intermediate nucleic acids. In some instances, the transposition reaction occurs at a transposition reaction temperature between 25-65° C. (e.g., between 35-65° C., between 40-65° C., between 45-65° C., between 50-65° C., between 55-65° C., between 60-65° C., between 25-60° C., between 25-55° C., between 25-50° C., between 25-45° C., between 25-40° C., between 25-35° C., between 25-30° C., between 40-50° C., or between 53-57° C.; e.g., at about 25° C., at about 30° C., about 35° C., about 40° C., about 45° C., about 50° C., about 53° C., about 54° C., about 55° C., about 56° C., about 57° C., about 60° C., or about 65° C.). In some instances, the transposition reaction occurs for a first reaction duration between 1 and 30 minutes (e.g., between 1 and 25 minutes, between 1 and 20 minutes, between 1 and 15 minutes, between 1 and 10 minutes, between 10 and 30 minutes, between 15 and 30 minutes, between 20 and 30 minutes, between 25 and 30 minutes, between 10 and 20 minutes, between 15 and 25 minutes; e.g., about 1 minutes, about 10 minutes, about 13 minutes, about 14 minutes, about 15 minutes, about 16 minutes, about 17 minutes, about 20 minutes, about 25 minutes, or about 30 minutes).

As depicted in FIG. 1C, after the transposition reaction, a polymerization reaction by the DNA polymerase will extend the 3′ ends of the intermediate nucleic acids to generate the sequencing oligonucleotides, depicted in FIG. 1D. In some instances, the polymerization reaction occurs at a polymerization reaction temperature between 55-95° C. (e.g., between 60-95° C., between 65-95° C., between 70-95° C., between 75-95° C., between 80-95° C., between 85-95° C., between 90-95° C., between 55-90° C., between 55-85° C., between 55-80° C., between 55-75° C., between 55-70° C., between 55-65° C., between 55-60° C., between 65-85° C., between 70-80° C., between 73-77° C.; e.g., at about 55° C., at about 60° C., at about 65° C., at about 70° C., at about 73° C., at about 74° C., at about 75° C., at about 76° C., at about 77° C., at about 80° C., at about 85° C., at about 90° C., at about 95° C.). In some instances, the polymerization reaction occurs for a second reaction duration between 1 and 60 minutes (e.g., between 1 and 55 minutes, between 1 and 50 minutes, between 1 and 45 minutes, between 1 and 40 minutes, between 1 and 35 minutes, between 1 and 30 minutes, between 1 and 25 minutes, between 1 and 20 minutes, between 1 and 15 minutes, between 1 and 10 minutes, between 10 and 60 minutes, between 15 and 60 minutes, between 20 and 60 minutes, between 25 and 60 minutes, between 30 and 60 minutes, between 35 and 60 minutes, between 40 and 60 minutes, between 45 and 60 minutes, between 50 and 60 minutes, between 55 and 60 minutes, between 15 and 35 minutes, between 30 and 50 minutes, between 10 and 20 minutes, between 20 and 40 minutes, or between 13 and 17 minutes; e.g., about 1 minute, about 5 minutes, about 10 minutes, about 13 minutes, about 14 minutes, about 15 minutes, about 16 minutes, about 17 minutes, about 20 minutes, about 25 minutes, about 30 minutes, about 35 minutes, about 40 minutes, about 45 minutes, about 50 minutes, about 55 minutes, or about 60 minutes). In some instances, the transposases dissociate as the first reaction vessel is heated to the polymerization temperature. In some instances, the DNA polymerase may be a thermostable DNA polymerase. In some instances, the DNA polymerase may be a hot-start DNA polymerase. In some instances, the hot-start DNA polymerase may contain an antibody or aptamer bound to the DNA polymerase, that is released when the hot-start DNA polymerase is heated to and incubated at a suitable temperature, which may be higher than the polymerization temperature.

In some instances, the sequencing oligonucleotides include (a) a nucleic acid sequence including a first universal primer region, a first adapter barcode region, a homologous sequence of a first nucleic acid fragment, a complement sequence of the second adapter barcode region, and a complement sequence of the second universal primer region, wherein a nucleic acid duplex including the first nucleic acid fragment and its complement is generated from the nucleic acid sample by transposase activity; and (b) the complement sequence thereof. In some instances, the sequencing oligonucleotides of the nucleic acid library may be further amplified through PCR to generate an amplified library. In some instances, the PCR reaction includes amplifying the sequencing oligonucleotides using a first universal primer and a second universal primer. In some instances, the first universal primer includes a first adapter priming region and the second universal primer includes a second adapter priming region, wherein the nucleic acid sequences of the first and second adapter priming regions are homologous to the nucleic acid sequences of the first and second universal primer regions, respectively.

In some instances, the nucleic acid sample, the first adapter oligonucleotide, the second adapter oligonucleotide, the pair of intermediate nucleic acids, and/or the sequencing oligonucleotides may include DNA. In some instances, the library, the first universal primer, and the second universal primer may include DNA. In some instances, the nucleic acid sample may include double-stranded DNA (dsDNA).

In some instances, the nucleic acid sample may include RNA. In some instances, the nucleic acid sample may include DNA and RNA. In some instances, an RNA sample can be transformed into a RNA/DNA duplex by reverse-transcription using a suitable reverse transcriptase, including, e.g., a Moloney murine leukemia virus (M-MLV) reverse transcriptase, an avian sarcoma-leukosis virus (ASLV) reverse transcriptase, and a human immunodeficiency virus (HIV) reverse transcriptase. Examples of Avian Sarcoma-Leukosis Virus (ASLV) reverse transcriptase include, e.g., Rous Sarcoma Virus (RSV) reverse transcriptase, Avian Reticuloendotheliosis Virus (REV-T) Helper Virus REV-A reverse transcriptase, Avian Erythroblastosis Virus (AEV) Helper Virus MCAV reverse transcriptase, Avian Myeloblastosis Virus (AMV) reverse transcriptase, Myeloblastosis Associated Virus (MAV) reverse transcriptase, Avian Myelocytomatosis Virus MC29 Helper Virus MCAV reverse transcriptase, Avian Sarcoma Virus Y73 Helper Virus YAV reverse transcriptase, Avian Sarcoma Virus UR2 Helper Virus UR2AV reverse transcriptase, and Rous Associated Virus (RAV) reverse transcriptase.

In some instances of the above methods for generating nucleic acid libraries including sequencing oligonucleotides, the DNA polymerase; the first synaptic complex, and the second synaptic complex; and magnesium ions (e.g., Mg²⁺) are provided in one or more kits of the invention described herein. In some instances, the DNA polymerase and magnesium ions are provided in a first composition, and the first synaptic complex and the second synaptic complex are provided in a second composition. In some instances, the DNA polymerase is provided in a first composition, the first synaptic complex and the second synaptic complex are provided in a second composition, and the magnesium ions are provided in a third composition. In some instances, the first, second, and optionally third compositions may be provided in a kit of the invention described herein.

In a particular example, a first reaction vessel may contain the following: about 3.2% (w/v) polyethylene glycol (PEG) 8000; about 0.11 μM Synaptic complex 1 (nE01); about 0.11 μM Synaptic complex 2 (PB037); about 3.125 μM Universal primer 1 (P5); about 3.125 μM Universal primer 2 (P7); about 1.5625 ng/μl Human DNA; about 14 mM TAPS, about pH 8.5; about 3 mM MgCl₂; about 2 ng/μl Hot-start DNA polymerase; about 25 mM NaCl; about 0.025 mM EDTA; about 12.5% (v/v) glycerol; about 0.25 mM dithiothreitol (DTT); and about 0.25% (v/v) TRITON® X-100. In this particular example, the first reaction vessel may be subject to the following thermocycler program:
Step 1 55° C. for about 15 minutes
Step 2 75° C. for about 15 minutes
Step 3 95° C. for about 2 minutes
Step 4 95° C. for about 25 seconds
Step 5 55° C. for about 30 seconds
Step 6 68° C. for about 1 minutes
Step 7 Return to Step 4, 10 times
Step 8 68° C. for about 5 minutes
Step 9 about 4° C. hold.

In another particular example, the first reaction vessel may contain: about 12.5 nM Synaptic complex 1; about 12.5 nM Synaptic complex 2; about 0.5 μM Universal primer 1 (P5); about 0.5 μM Universal primer 2 (P7); about 0.5-4 ng/μL (variable input) pUC19 DNA (plasmid DNA); about 10 mM TAPS, pH 8.5; about 2.5 mM MgCl₂; about 0.02 Units/μL Hot-start DNA polymerase; about 1×Polymerase Buffer; and about 0.2 mM dNTPs; and about 7% (v/v) DMSO. In this particular example, the first reaction vessel may be subject to the following thermocycler program:
Step 1 about 55° C. for about 5 minutes
Step 2 about 75° C. for about 5 minutes
Step 3 about 79° C. for about 5 minutes
Step 4 about 83° C. for about 5 minutes
Step 5 about 98° C. for about 3 minutes
Step 6 about 98° C. for about 15 seconds
Step 7 about 64° C. for about 30 seconds
Step 8 about 72° C. for about 1 minute
Step 9 Return to Step 6, 12 times
Step 10 about 72° C. for about 15 minutes
Step 11 about 4° C. hold.
   Methods for Generating Nucleic Acid Libraries with Amplifier Oligonucleotides

The invention provides methods for the generation of nucleic acid libraries having amplicons using adapter oligonucleotides and amplifier oligonucleotides. In some instances, the methods include generating the nucleic acid libraries using (a) a DNA polymerase; (b) a first synaptic complex including a first transposase and a first adapter oligonucleotide, and a second synaptic complex including a second transposase and a second adapter oligonucleotide; and (c) magnesium ions (e.g., Mg²⁺). In some instances, each adapter oligonucleotide includes an adapter priming region. In some instances, each adapter oligonucleotide includes an adapter priming region and an adapter barcode region. The method may further include using (i) a first amplifier oligonucleotide including a first universal primer region and a first amplifier priming region; and (ii) a second amplifier oligonucleotide including a second universal primer region and a second amplifier priming region, wherein the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

The sequencing oligonucleotides may be generated in a single reaction vessel through a method that includes a transposition reaction, a polymerization reaction, and a PCR reaction. The method includes first combining the DNA polymerase, the first synaptic complex and the second synaptic complex, magnesium ions (e.g., Mg²⁺), the first and second amplifier oligonucleotides, and the nucleic acid sample in a first reaction vessel. The transposases of the synaptic complexes will fragment the nucleic acid sample into nucleic acid fragments and add the adapter oligonucleotide sequences to the 5′ ends of the two strands of a nucleic acid duplex containing a nucleic acid fragment and its complement sequence in a transposition reaction to generate intermediate nucleic acids. In some instances, the transposition reaction occurs at a transposition reaction temperature between 25-65° C. (e.g., between 35-65° C., between 40-65° C., between 45-65° C., between 50-65° C., between 55-65° C., between 60-65° C., between 25-60° C., between 25-55° C., between 25-50° C., between 25-45° C., between 25-40° C., between 25-35° C., between 25-30° C., between 40-50° C., or between 53-57° C.; e.g., at about 25° C., at about 30° C., about 35° C., about 40° C., about 45° C., about 50° C., about 53° C., about 54° C., about 55° C., about 56° C., about 57° C., about 60° C., or about 65° C.). In some instances, the transposition reaction occurs for a first reaction duration between 1 and 30 minutes (e.g., between 1 and 25 minutes, between 1 and 20 minutes, between 1 and 15 minutes, between 1 and 10 minutes, between 1 and 5 minutes, between 5 and 10 minutes, between 5 and 20 minutes, between 5 and 30 minutes, between 10 and 30 minutes, between 15 and 30 minutes, between 20 and 30 minutes, between 25 and 30 minutes, between 10 and 20 minutes, between 15 and 25 minutes; e.g., about 1 minute, about 5 minutes, about 10 minutes, about 13 minutes, about 14 minutes, about 15 minutes, about 16 minutes, about 17 minutes, about 20 minutes, about 25 minutes, or about 30 minutes).

After the transposition reaction, a polymerization reaction by the DNA polymerase of the first composition will extend the 3′ ends of the intermediate nucleic acids to generate full duplexes. In some instances, the polymerization reaction occurs at a polymerization reaction temperature between 55-95° C. (e.g., between 60-95° C., between 65-95° C., between 70-95° C., between 75-95° C., between 80-95° C., between 85-95° C., between 90-95° C., between 55-90° C., between 55-85° C., between 55-80° C., between 55-75° C., between 55-70° C., between 55-65° C., between 55-60° C., between 65-85° C., between 70-80° C., between 73-77° C.; e.g., at about 55° C., at about 60° C., at about 65° C., at about 70° C., at about 73° C., at about 74° C., at about 75° C., at about 76° C., at about 77° C., at about 80° C., at about 85° C., at about 90° C., at about 95° C.). In some instances, the transposases dissociate as the first reaction vessel is heated to the polymerization temperature. In some instances, the DNA polymerase may be a thermostable DNA polymerase. In some instances, the DNA polymerase may be a hot-start DNA polymerase. In some instances, the hot-start DNA polymerase may contain an antibody or aptamer bound to the DNA polymerase, that is released when the hot-start DNA polymerase heated to and incubated at a suitable temperature, which may or may not be higher than the optimal polymerization temperature. A PCR reaction using the amplifier oligonucleotides as primers then generates the amplicons of the nucleic acid library.

In some instances, the amplicons include (a) a nucleic acid sequence including a first universal primer region, a first amplifier priming region, a homologous sequence of a first nucleic acid fragment, a complement sequence of the second amplifier priming region, and a complement sequence of the second universal primer region; and (b) the complement sequence thereof. In other instances, i.e., when the adapter oligonucleotides include both an adapter primer region and an adapter barcode region, the amplicons include (a) a nucleic acid sequence including a first universal primer region, a first amplifier priming region, a first adapter barcode region, a homologous sequence of a first nucleic acid fragment, a complement sequence of the second adapter barcode region, a complement sequence of the second amplifier priming region, and a complement sequence of the second universal primer region; and (b) the complement sequence thereof. In some instances, a nucleic acid duplex including the first nucleic acid fragment and its complement is generated from the nucleic acid sample by transposase activity. In some instances, the PCR reaction produces amplicons from the intermediate nucleic acids using the amplifier oligonucleotides. In some instances, the PCR reaction includes 1-35 cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 cycles).

In some instances, the nucleic acid sample, the first adapter oligonucleotide, the second adapter oligonucleotide, the first amplifier oligonucleotide, the second amplifier oligonucleotide, the intermediate nucleic acids, and/or the amplicons may include DNA. In some instances, the nucleic acid sample may include double-stranded DNA.

In some instances, the nucleic acid sample may include RNA. In some instances, the nucleic acid sample may include DNA and RNA. In some instances, an RNA sample can be transformed into an RNA/DNA duplex by reverse-transcription using a suitable reverse transcriptase, including, e.g., a Moloney murine leukemia virus (M-MLV) reverse transcriptase, a human immunodeficiency virus (HIV) reverse transcriptase, and an avian sarcoma-leukosis virus (ASLV) reverse transcriptase. Avian Sarcoma-Leukosis Virus (ASLV) reverse transcriptase includes, but is not limited to, Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase.

In some instances of the above methods for generating nucleic acid libraries including amplicons, the DNA polymerase; the first synaptic complex, and the second synaptic complex; and magnesium ions (e.g., Mg²⁺) are provided in one or more compositions of the invention described herein. In some instances, the DNA polymerase and magnesium ions are provided in a first composition, and the first synaptic complex and the second synaptic complex are provided in a second composition. In some instances, the DNA polymerase is provided in a first composition, the first synaptic complex and the second synaptic complex are provided in a second composition, and the magnesium ions are provided in a third composition. In some instances, the first, second, and optionally third compositions may be provided in a kit of the invention described herein.

Methods for Generating Nucleic Acid Libraries with Barcoded Amplifier Oligonucleotides

The invention provides methods for the generation of nucleic acid libraries having amplicons using adapter oligonucleotides and barcoded amplifier oligonucleotides. In some instances, the methods include generating the nucleic acid libraries using (a) a DNA polymerase; (b) a first synaptic complex including a first transposase and a first adapter oligonucleotide, and a second synaptic complex including a second transposase and a second adapter oligonucleotide; and (c) magnesium ions (e.g., Mg²⁺). In some instances, each adapter oligonucleotide includes an adapter priming region. The method may further include using (i) a first amplifier oligonucleotide including a first universal primer region, a first amplifier barcode region, and a first amplifier priming region; and (ii) a second amplifier oligonucleotide including a second universal primer region, a second amplifier barcode region, and a second amplifier priming region, wherein the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

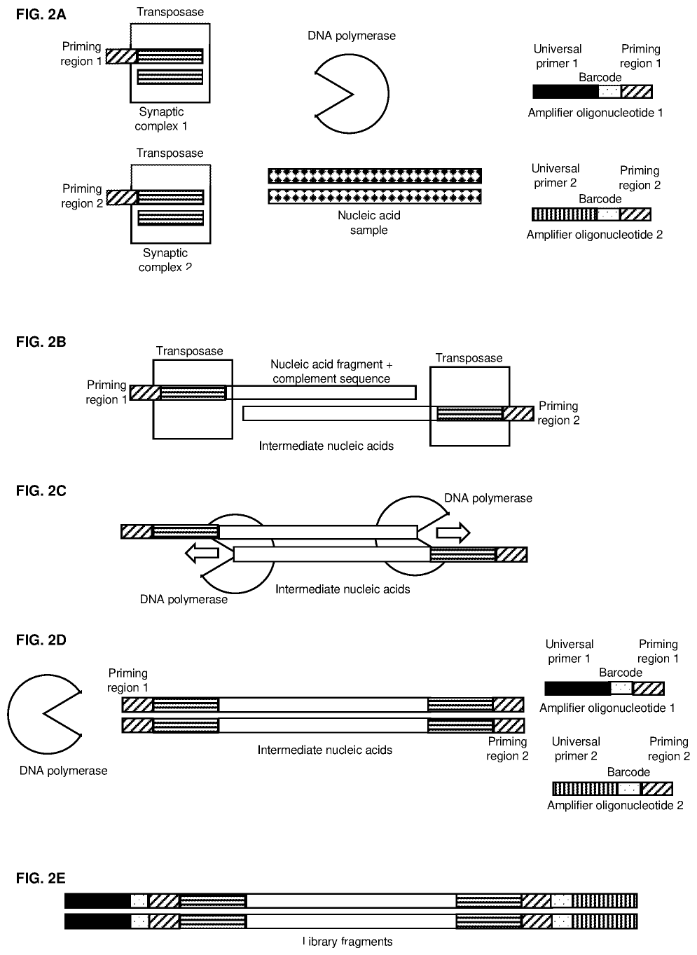

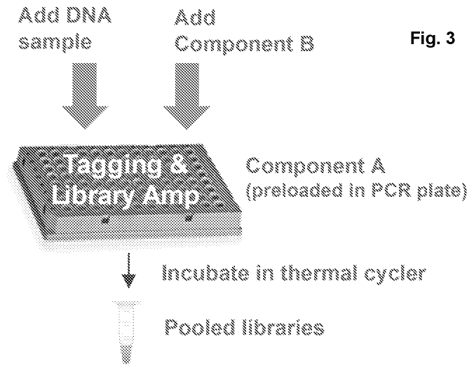

As depicted in FIGS. 2A-2E, the sequencing oligonucleotides may be generated in a single reaction vessel through a method that includes a transposition reaction, a polymerization reaction, and a PCR reaction. As depicted in FIG. 2A, the method includes first combining the DNA polymerase, the first synaptic complex and the second synaptic complex, magnesium ions (e.g., Mg²⁺), the first and second amplifier oligonucleotides, and the nucleic acid sample in a first reaction vessel. As depicted in FIG. 2B, the transposases of the synaptic complexes will fragment the nucleic acid sample into nucleic acid fragments, and add the adapter oligonucleotide sequences to the 5′ ends of the two strands of a nucleic acid duplex containing a nucleic acid fragment and its complement sequence in a transposition reaction to generate intermediate nucleic acids. In some instances, the transposition reaction occurs at a transposition reaction temperature between 25-65° C. (e.g., between 35-65° C., between 40-65° C., between 45-65° C., between 50-65° C., between 55-65° C., between 60-65° C., between 25-60° C., between 25-55° C., between 25-50° C., between 25-45° C., between 25-40° C., between 25-35° C., between 25-30° C., between 40-50° C., or between 53-57° C.; e.g., at about 25° C., at about 30° C., about 35° C., about 40° C., about 45° C., about 50° C., about 53° C., about 54° C., about 55° C., about 56° C., about 57° C., about 60° C., or about 65° C.). In some instances, the transposition reaction occurs for a first reaction duration between 1 and 30 minutes (e.g., between 1 and 25 minutes, between 1 and 20 minutes, between 1 and 15 minutes, between 1 and 10 minutes, between 1 and 5 minutes, between 5 and 10 minutes, between 5 and 20 minutes, between 5 and 30 minutes, between 10 and 30 minutes, between 15 and 30 minutes, between 20 and 30 minutes, between 25 and 30 minutes, between 10 and 20 minutes, between 15 and 25 minutes; e.g., about 1 minute, about 5 minutes, about 10 minutes, about 13 minutes, about 14 minutes, about 15 minutes, about 16 minutes, about 17 minutes, about 20 minutes, about 25 minutes, or about 30 minutes).

As depicted in FIG. 2C, after the transposition reaction, a polymerization reaction by the DNA polymerase of the first composition will extend the 3′ ends of the intermediate nucleic acids to generate full duplexes, depicted in FIG. 2D. In some instances, the polymerization reaction occurs at a polymerization reaction temperature between 55-95° C. (e.g., between 60-95° C., between 65-95° C., between 70-95° C., between 75-95° C., between 80-95° C., between 85-95° C., between 90-95° C., between 55-90° C., between 55-85° C., between 55-80° C., between 55-75° C., between 55-70° C., between 55-65° C., between 55-60° C., between 65-85° C., between 70-80° C., between 73-77° C.; e.g., at about 55° C., at about 60° C., at about 65° C., at about 70° C., at about 73° C., at about 74° C., at about 75° C., at about 76° C., at about 77° C., at about 80° C., at about 85° C., at about 90° C., at about 95° C.). In some instances, the transposases dissociate as the first reaction vessel is heated to the polymerization temperature. In some instances, the DNA polymerase may be a thermostable DNA polymerase. In some instances, the DNA polymerase may be a hot-start DNA polymerase. In some instances, the hot-start DNA polymerase may contain an antibody or aptamer bound to the DNA polymerase, that is released when the hot-start DNA polymerase heated to and incubated at a suitable temperature, which may or may not be higher than the optimal polymerization temperature. As depicted in FIG. 2E, a PCR reaction using the amplifier oligonucleotides as primers then generates the amplicons of the nucleic acid library.

In some instances, the amplicons include (a) a nucleic acid sequence including a first universal primer region, a first amplifier barcode region, a first amplifier priming region, a homologous sequence of a first nucleic acid fragment, a complement sequence of the second amplifier priming region, a complement sequence of the second amplifier barcode region, and a complement sequence of the second universal primer region; and (b) the complement sequence thereof. In some instances, a nucleic acid duplex including the first nucleic acid fragment and its complement is generated from the nucleic acid sample by transposase activity. In some instances, the intermediate nucleic acid depicted in FIG. 2D is the template for the PCR reaction depicted in FIG. 2E. In some instances, the PCR reaction produces amplicons from the intermediate nucleic acids using the amplifier oligonucleotides. In some instances, the PCR reaction includes 1-35 cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 cycles).

In some instances, the nucleic acid sample, the first adapter oligonucleotide, the second adapter oligonucleotide, the first amplifier oligonucleotide, the second amplifier oligonucleotide, the intermediate nucleic acids, and/or the amplicons may include DNA. In some instances, the nucleic acid sample may include double-stranded DNA.

In some instances, the nucleic acid sample may include RNA. In some instances, the nucleic acid sample may include DNA and RNA. In some instances, an RNA sample can be transformed into an RNA/DNA duplex by reverse-transcription using a suitable reverse transcriptase, including, e.g., a Moloney murine leukemia virus (M-MLV) reverse transcriptase, a human immunodeficiency virus (HIV) reverse transcriptase, and an avian sarcoma-leukosis virus (ASLV) reverse transcriptase. Avian Sarcoma-Leukosis Virus (ASLV) reverse transcriptase includes, but is not limited to, Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase.

In some instances of the above methods for generating nucleic acid libraries including amplicons, the DNA polymerase; the first synaptic complex, and the second synaptic complex; and magnesium ions (e.g., Mg²⁺) are provided in one or more compositions of the invention described herein. In some instances, the DNA polymerase and magnesium ions are provided in a first composition, and the first synaptic complex and the second synaptic complex are provided in a second composition. In some instances, the DNA polymerase is provided in a first composition, the first synaptic complex and the second synaptic complex are provided in a second composition, and the magnesium ions are provided in a third composition. In some instances, the first, second, and optionally third compositions may be provided in a kit of the invention described herein.

### Methods for Sequencing

The methods of the invention may further include determining the nucleic acid sequences of the sequencing oligonucleotides or amplicons through nucleic acid sequencing (e.g., next-generation sequencing (NGS)) or other methods known in the art. In some instances, sequencing can be performed by various systems that are currently available, e.g., a sequencing system by Pacific Biosciences (PACBIO®), ILLUMINA®, Oxford NANOPORE®, Genapsys@, or ThermoFisher (ION TORRENT®). Alternatively or in addition, sequencing may be performed using nucleic acid amplification, sequencing-by-ligation (e.g., SOLID), sequencing-by-synthesis, polymerase chain reaction (PCR) (e.g., digital PCR, quantitative PCR, or real time PCR), or isothermal amplification. In some instances, the sequencing oligonucleotides and amplicons described herein can be uniquely identified based on the nucleic acid sequences of the nucleic acid fragment and the nucleic acid sequences of the adapter barcode regions or amplifier barcode regions of the sequencing oligonucleotides or amplicons, respectively. The invention further includes data generated by nucleic acid sequencing, as well as methods for generating and analyzing such sequence data, and reaction mixtures used in and formed by such methods.

### EXAMPLES

The invention is described by the following non-limiting examples.

### Example 1. “One-Step” Nucleic Acid Library Preparation Method

For the one-step reaction, the following components were added to a single reaction vessel (e.g., 96-well plate) in a reaction volume of 32 μl at the final concentrations shown in Table 1. The reaction vessel was placed into a thermocycler running the program described below in Table 2.

TABLE 1
One-step reaction set-up

| Reaction Component | Final concentration | |
|---|---|---|
| PEG 8000 | 3.2% | (w/v) |
| Synaptic complex 1 (nE01) | 0.11 | μM |
| Synaptic complex 2 (PB037) | 0.11 | μM |
| Universal primer 1 (P5) | 3.125 | μM |
| Universal primer 2 (P7) | 3.125 | μM |
| Human DNA | 1.5625 | ng/μl |
| TAPS, pH 8.5 | 14 | mM |
| MgCl₂ | 3 | mM |
| Hot-start DNA polymerase | 2 | ng/μl |
| NaCl | 25 | mM |
| EDTA | 0.025 | mM |
| Glycerol | 12.5% | (v/v) |
| DTT | 0.25 | mM |
| TRITON ® X-100 | 0.25% | (v/v) |

TABLE 2
Thermocycler Program for the One-step Reaction

| Step | Condition | Purpose |
|---|---|---|
| 1 | 55° C. for 15 min | Transposition reaction (tagging) |
| 2 | 75° C. for 15 min | Releases Tn5 transposase |
| | | and activates hot-start DNA |
| | | polymerase to fill-in adapter |
| 3 | 95° C. for 2 min | Initial denaturation |
| 4 | 95° C. for 25 sec | Denaturation |
| 5 | 55° C. for 30 sec | Annealing |
| 6 | 68° C. for 1 min | Extension |
| 7 | Return to Step 4, 10 times | Cycling |
| 8 | 68° C. for 5 min | Final Extension |
| 9 | 4° C. hold | Hold |

The following nucleic acid sequences were used in the above protocol for the preparation of the amplified nucleic acid library.

SEQ ID 1: Full length first adapter oligonucleotide:
CAA GCA GAA GAC GGC ATA CGA GAT AGT CAC
CAG TCT CGT GGG CTC GGA GAT GTG TAT AAG AGA CAG
SEQ ID 2: Full length second adapter oligonucleotide:
AAT GAT ACG GCG ACC ACC GAG ATC TAC
ACA AGG AGT ATC GTC GGC AGC GTC AGA TGT GTA TAA GAG ACA G
SEQ ID 3: ME′ (5′-phosphorylated complement of the transposase mosaic end sequence):
/5Phos/CTG TCT CTT ATA CAC ATC T/3InvdT/
SEQ ID 4: Universal primer 1:
CAA GCA GAA GAC GGC ATA CGA G
SEQ ID 5: Universal primer 2:
AAT GAT ACG GCG ACC ACC GAG
SEQ ID 6: Amplifier oligonucleotide 1:
CAA GCA GAA GAC GGC ATA CGA GAT GGA AGA GAT AGT
CTC GTG GGC TCG G
SEQ ID 7: Amplifier oligonucleotide 2:
AAT GAT ACG GCG ACC ACC GAG ATC TAC ACC CAC AAC
TTA TCG TCG GCA GCG TC
SEQ ID 8: First adapter oligonucleotide:
TCG TCG GCA GCG TCA GAT GTG TAT AAG AGA CAG
SEQ ID 9: Second adapter oligonucleotide:
GTC TCG TGG GCT CGG AGA TGT GTA TAA GAG ACA G

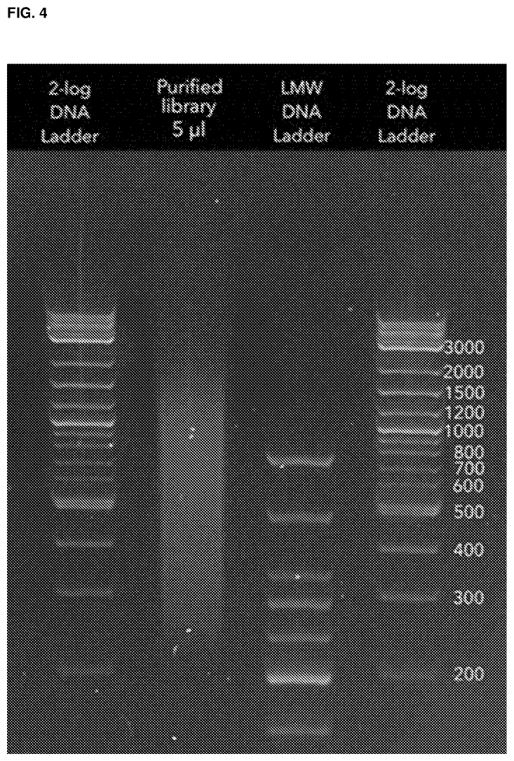

The amplified nucleic acid library resulting from the one-step reaction from an exemplary DNA sample was purified with 0.8 volumetric equivalents of MAGwise™ paramagnetic beads according to the manufacturer's instructions (seqWell, Inc.). An agarose gel of the purified library is shown in FIG. 4, demonstrating a broad range of DNA fragment sizes in the amplified sequencing library. The nucleic acid library was sequenced on a MiSeq® sequencer (ILLUMINA®).

### Results

A nucleic acid library prepared from a 50 ng human DNA sample using the one-step library preparation method described above was sequenced using an Illumina MiSeq® sequencer with a v3 sequencing kit. Of the 33,340,460 total read pairs, 100% were PF (passing filter) reads, and 97.7% aligned to the human hg38 reference sequence. Additionally, the amplified library exhibited high diversity, wherein of the ˜32 million read pairs analyzed, only about 1.3% were duplicate reads. The amplified fragments averaged 261±159 bp (mean±std. dev.) in length.

### Example 2. “One-Step” Nucleic Acid Library Preparation Method for Plasmids

For preparing NGS libraries from plasmid DNA (pUC19) in one-step reactions, the following components were added to a single reaction vessel (e.g., a 96-well plate) in a reaction volume of 16 μl at the final concentrations shown in Table 3. The reaction vessel was placed into a thermocycler running the program described in Table 4.

TABLE 3
One-step reaction set-up

| Reaction Component | Final concentration | |
|---|---|---|
| i7 Synaptic complex 1 | 12.5 | nM |
| i5 Synaptic complex 2 | 12.5 | nM |
| Universal primer 1 (P5) | 0.5 | μM |
| Universal primer 2 (P7) | 0.5 | μM |
| pUC19 DNA | 0.5-4 ng/μL (variable input) | |
| TAPS, pH 8.5 | 10 | mM |
| MgCl₂ | 2.5 | mM |
| Hot-start DNA polymerase | 0.02 | Units/μL |
| Polymerase Buffer | 1X | |
| dNTPs | 0.2 | mM |
| DMSO | 7% | |
| Total Reaction Volume | 16 | μL |

TABLE 4
Thermocycler Program for the one-step reaction

| Step | Condition | Purpose |
|---|---|---|
| 1 | 55° C. for 5 minutes | Transposition reaction (tagging) |
| 2 | 75° C. for 5 minutes | Releases Tn5 transposase |
| | | and activates hot-start DNA |
| | | polymerase to fill-in adapters |
| 3 | 79° C. for 5 minutes | Releases Tn5 transposase |
| | | and activates hot-start DNA |
| | | polymerase to fill-in adapters |
| 4 | 83° C. for 5 minutes | Releases Tn5 transposase |
| | | and activates hot-start DNA |
| | | polymerase to fill-in adapters |
| 5 | 98° C. for 3 minutes | Initial denaturation |
| 6 | 98° C. for 15 seconds | Denaturation |
| 7 | 64° C. for 30 seconds | Annealing |
| 8 | 72° C. for 1 minute | Extension |
| 9 | Return to Step 6, 12 times | Cycling |
| 10 | 72° C. for 15 minutes | Final Extension |
| 11 | 4° C. hold | Hold |

The following nucleic acid sequences were used in Example 2 to make synaptic complexes, tag plasmid DNA, and amplify nucleic acid libraries in one-step reactions:

SEQ ID 10:
CAA GCA GAA GAC GGC ATA CGA GAT GAG TTA GTT GGT CTC GTG GGC TCG GAG
ATG TGT ATA AGA GAC AG
SEQ ID 11:
CAA GCA GAA GAC GGC ATA CGA GAT AAT CCA GTA CGT CTC GTG GGC TCG GAG
ATG TGT ATA AGA GAC AG
SEQ ID 12:
CAA GCA GAA GAC GGC ATA CGA GAT TCC TCC GAT GGT CTC GTG GGC TCG GAG
ATG TGT ATA AGA GAC AG
SEQ ID 13:
CAA GCA GAA GAC GGC ATA CGA GAT CCA TCA GAA TGT CTC GTG GGC TCG GAG
ATG TGT ATA AGA GAC AG
SEQ ID 14:
CAA GCA GAA GAC GGC ATA CGA GAT GGT AAC GAT AGT CTC GTG GGC TCG GAG
ATG TGT ATA AGA GAC AG
SEQ ID 15:
CAA GCA GAA GAC GGC ATA CGA GAT TAA CAG ACC AGT CTC GTG GGC TCG GAG
ATG TGT ATA AGA GAC AG
SEQ ID 16:
CAA GCA GAA GAC GGC ATA CGA GAT CAT GTT GGC AGT CTC GTG GGC TCG GAG
ATG TGT ATA AGA GAC AG
SEQ ID 17:
CAA GCA GAA GAC GGC ATA CGA GAT CGT GTA ATG AGT CTC GTG GGC TCG GAG
ATG TGT ATA AGA GAC AG
SEQ ID 18:
CAA GCA GAA GAC GGC ATA CGA GAT GTC AGG TTA TGT CTC GTG GGC TCG GAG
ATG TGT ATA AGA GAC AG
SEQ ID 19:
CAA GCA GAA GAC GGC ATA CGA GAT TGG AAC CGC AGT CTC GTG GGC TCG GAG
ATG TGT ATA AGA GAC AG
SEQ ID 20:
CAA GCA GAA GAC GGC ATA CGA GAT GGC AAG TAT TGT CTC GTG GGC TCG GAG
ATG TGT ATA AGA GAC AG
SEQ ID 21:
CAA GCA GAA GAC GGC ATA CGA GAT TTC CGC GAT TGT CTC GTG GGC TCG GAG
ATG TGT ATA AGA GAC AG
SEQ ID 22:
CAA GCA GAA GAC GGC ATA CGA GAT TCG CGT TCT GGT CTC GTG GGC TCG GAG
ATG TGT ATA AGA GAC AG
SEQ ID 23:
CAA GCA GAA GAC GGC ATA CGA GAT CCT TGG TTC AGT CTC GTG GGC TCG GAG
ATG TGT ATA AGA GAC AG
SEQ ID 24:
CAA GCA GAA GAC GGC ATA CGA GAT CTG ACT AAC AGT CTC GTG GGC TCG GAG
ATG TGT ATA AGA GAC AG
SEQ ID 25:
CAA GCA GAA GAC GGC ATA CGA GAT TTA CCT ACT GGT CTC GTG GGC TCG GAG
ATG TGT ATA AGA GAC AG
SEQ ID 26:
CAA GCA GAA GAC GGC ATA CGA GAT GAA GTA CCT GGT CTC GTG GGC TCG GAG
ATG TGT ATA AGA GAC AG
SEQ ID 27:
CAA GCA GAA GAC GGC ATA CGA GAT CAT ATT CCT GGT CTC GTG GGC TCG GAG
ATG TGT ATA AGA GAC AG
SEQ ID 28:
CAA GCA GAA GAC GGC ATA CGA GAT ATT GTG CGT TGT CTC GTG GGC TCG GAG
ATG TGT ATA AGA GAC AG
SEQ ID 29:
CAA GCA GAA GAC GGC ATA CGA GAT CCT TCT CAA TGT CTC GTG GGC TCG GAG
ATG TGT ATA AGA GAC AG
SEQ ID 30:
CAA GCA GAA GAC GGC ATA CGA GAT AAG GTG CAA TGT CTC GTG GGC TCG GAG
ATG TGT ATA AGA GAC AG
SEQ ID 31:
CAA GCA GAA GAC GGC ATA CGA GAT GTA TCC ACT TGT CTC GTG GGC TCG GAG
ATG TGT ATA AGA GAC AG
SEQ ID 32:
CAA GCA GAA GAC GGC ATA CGA GAT GCA TCT TAT CGT CTC GTG GGC TCG GAG
ATG TGT ATA AGA GAC AG
SEQ ID 33:
CAA GCA GAA GAC GGC ATA CGA GAT TGT CGA GGT AGT CTC GTG GGC TCG GAG
ATG TGT ATA AGA GAC AG
SEQ ID 34:
CAA GCA GAA GAC GGC ATA CGA GAT TGG CTC GGT TGT CTC GTG GGC TCG GAG
ATG TGT ATA AGA GAC AG
SEQ ID 35:
CAA GCA GAA GAC GGC ATA CGA GAT CTT GAA TCC AGT CTC GTG GGC TCG GAG
ATG TGT ATA AGA GAC AG
SEQ ID 36:
CAA GCA GAA GAC GGC ATA CGA GAT GTA TCT GTT GGT CTC GTG GGC TCG GAG
ATG TGT ATA AGA GAC AG
SEQ ID 37:
CAA GCA GAA GAC GGC ATA CGA GAT GAG TTA CCT TGT CTC GTG GGC TCG GAG
ATG TGT ATA AGA GAC AG
SEQ ID 38:
CAA GCA GAA GAC GGC ATA CGA GAT CGT ATT GTC AGT CTC GTG GGC TCG GAG
ATG TGT ATA AGA GAC AG
SEQ ID 39:
CAA GCA GAA GAC GGC ATA CGA GAT ATG CAC AAT CGT CTC GTG GGC TCG GAG
ATG TGT ATA AGA GAC AG
SEQ ID 40:
CAA GCA GAA GAC GGC ATA CGA GAT AAT GGT GTG CGT CTC GTG GGC TCG GAG
ATG TGT ATA AGA GAC AG
SEQ ID 41:
CAA GCA GAA GAC GGC ATA CGA GAT GGA ATA ACG AGT CTC GTG GGC TCG GAG
ATG TGT ATA AGA GAC AG
SEQ ID 42:
CAA GCA GAA GAC GGC ATA CGA GAT AAT GAC GTT GGT CTC GTG GGC TCG GAG
ATG TGT ATA AGA GAC AG
SEQ ID 43:
CAA GCA GAA GAC GGC ATA CGA GAT CAT CGT TCT TGT CTC GTG GGC TCG GAG
ATG TGT ATA AGA GAC AG
SEQ ID 44:
CAA GCA GAA GAC GGC ATA CGA GAT GTA ATT GCA GGT CTC GTG GGC TCG GAG
ATG TGT ATA AGA GAC AG
SEQ ID 45:
CAA GCA GAA GAC GGC ATA CGA GAT ATC ATC GGC GGT CTC GTG GGC TCG GAG
ATG TGT ATA AGA GAC AG
SEQ ID 46:
CAA GCA GAA GAC GGC ATA CGA GAT GTC TTG TCC GGT CTC GTG GGC TCG GAG
ATG TGT ATA AGA GAC AG
SEQ ID 47:
CAA GCA GAA GAC GGC ATA CGA GAT GAG GAG TAC GGT CTC GTG GGC TCG GAG
ATG TGT ATA AGA GAC AG
SEQ ID 48:
CAA GCA GAA GAC GGC ATA CGA GAT AAC TCA TGC AGT CTC GTG GGC TCG GAG
ATG TGT ATA AGA GAC AG
SEQ ID 49:
CAA GCA GAA GAC GGC ATA CGA GAT GCT CCA GAT TGT CTC GTG GGC TCG GAG
ATG TGT ATA AGA GAC AG
SEQ ID 50:
CAA GCA GAA GAC GGC ATA CGA GAT TCT GAC CAA TGT CTC GTG GGC TCG GAG
ATG TGT ATA AGA GAC AG
SEQ ID 51:
CAA GCA GAA GAC GGC ATA CGA GAT AAC AGG TCT TGT CTC GTG GGC TCG GAG
ATG TGT ATA AGA GAC AG
SEQ ID 52:
CAA GCA GAA GAC GGC ATA CGA GAT TTC ATT AGC GGT CTC GTG GGC TCG GAG
ATG TGT ATA AGA GAC AG
SEQ ID 53:
CAA GCA GAA GAC GGC ATA CGA GAT ATG CAC TCT GGT CTC GTG GGC TCG GAG
ATG TGT ATA AGA GAC AG
SEQ ID 54:
CAA GCA GAA GAC GGC ATA CGA GAT TCA GCT TAC CGT CTC GTG GGC TCG GAG
ATG TGT ATA AGA GAC AG
SEQ ID 55:
CAA GCA GAA GAC GGC ATA CGA GAT CAC TGA ATA CGT CTC GTG GGC TCG GAG
ATG TGT ATA AGA GAC AG
SEQ ID 56:
CAA GCA GAA GAC GGC ATA CGA GAT AAC GTA GCG CGT CTC GTG GGC TCG GAG
ATG TGT ATA AGA GAC AG
SEQ ID 57:
CAA GCA GAA GAC GGC ATA CGA GAT GCT ATG ACA AGT CTC GTG GGC TCG GAG
ATG TGT ATA AGA GAC AG
SEQ ID 58:
CAA GCA GAA GAC GGC ATA CGA GAT TCC GCA ACA TGT CTC GTG GGC TCG GAG
ATG TGT ATA AGA GAC AG
SEQ ID 59:
CAA GCA GAA GAC GGC ATA CGA GAT GGT TAG CAT TGT CTC GTG GGC TCG GAG
ATG TGT ATA AGA GAC AG
SEQ ID 60:
CAA GCA GAA GAC GGC ATA CGA GAT TAC GTG TTA CGT CTC GTG GGC TCG GAG
ATG TGT ATA AGA GAC AG
SEQ ID 61:
CAA GCA GAA GAC GGC ATA CGA GAT TCC GTA TAT CGT CTC GTG GGC TCG GAG
ATG TGT ATA AGA GAC AG
SEQ ID 62:
CAA GCA GAA GAC GGC ATA CGA GAT CCT CTC GGA TGT CTC GTG GGC TCG GAG
ATG TGT ATA AGA GAC AG
SEQ ID 63:
CAA GCA GAA GAC GGC ATA CGA GAT GTG TGA ATT GGT CTC GTG GGC TCG GAG
ATG TGT ATA AGA GAC AG
SEQ ID 64:
CAA GCA GAA GAC GGC ATA CGA GAT GAA CTG ACA AGT CTC GTG GGC TCG GAG
ATG TGT ATA AGA GAC AG
SEQ ID 65:
CAA GCA GAA GAC GGC ATA CGA GAT AAT GGC GTA TGT CTC GTG GGC TCG GAG
ATG TGT ATA AGA GAC AG
SEQ ID 66:
CAA GCA GAA GAC GGC ATA CGA GAT GTT ATT GGT GGT CTC GTG GGC TCG GAG
ATG TGT ATA AGA GAC AG
SEQ ID 67:
CAA GCA GAA GAC GGC ATA CGA GAT TTG TGG ACG CGT CTC GTG GGC TCG GAG
ATG TGT ATA AGA GAC AG
SEQ ID 68:
CAA GCA GAA GAC GGC ATA CGA GAT AAG CGC ACA TGT CTC GTG GGC TCG GAG
ATG TGT ATA AGA GAC AG
SEQ ID 69:
CAA GCA GAA GAC GGC ATA CGA GAT GCA CTA GAT AGT CTC GTG GGC TCG GAG
ATG TGT ATA AGA GAC AG
SEQ ID 70:
CAA GCA GAA GAC GGC ATA CGA GAT CAT TGT AGG CGT CTC GTG GGC TCG GAG
ATG TGT ATA AGA GAC AG
SEQ ID 71:
CAA GCA GAA GAC GGC ATA CGA GAT AAC TGA GCT TGT CTC GTG GGC TCG GAG
ATG TGT ATA AGA GAC AG
SEQ ID 72:
CAA GCA GAA GAC GGC ATA CGA GAT TCT AAG CGT AGT CTC GTG GGC TCG GAG
ATG TGT ATA AGA GAC AG
SEQ ID 73:
CAA GCA GAA GAC GGC ATA CGA GAT CCT AGT GGA TGT CTC GTG GGC TCG GAG
ATG TGT ATA AGA GAC AG
SEQ ID 74:
CAA GCA GAA GAC GGC ATA CGA GAT AAG ACG GAC AGT CTC GTG GGC TCG GAG
ATG TGT ATA AGA GAC AG
SEQ ID 75:
CAA GCA GAA GAC GGC ATA CGA GAT GTT ACT CAT GGT CTC GTG GGC TCG GAG
ATG TGT ATA AGA GAC AG
SEQ ID 76:
CAA GCA GAA GAC GGC ATA CGA GAT ATT CGC GCA GGT CTC GTG GGC TCG GAG
ATG TGT ATA AGA GAC AG
SEQ ID 77:
CAA GCA GAA GAC GGC ATA CGA GAT CAT ACT ACA GGT CTC GTG GGC TCG GAG
ATG TGT ATA AGA GAC AG
SEQ ID 78:
CAA GCA GAA GAC GGC ATA CGA GAT TGT ACG GCT CGT CTC GTG GGC TCG GAG
ATG TGT ATA AGA GAC AG
SEQ ID 79:
CAA GCA GAA GAC GGC ATA CGA GAT CCT AAG AAT GGT CTC GTG GGC TCG GAG
ATG TGT ATA AGA GAC AG
SEQ ID 80:
CAA GCA GAA GAC GGC ATA CGA GAT TTG TCG GTC AGT CTC GTG GGC TCG GAG
ATG TGT ATA AGA GAC AG
SEQ ID 81:
CAA GCA GAA GAC GGC ATA CGA GAT CGG TTA CAC AGT CTC GTG GGC TCG GAG
ATG TGT ATA AGA GAC AG
SEQ ID 82:
CAA GCA GAA GAC GGC ATA CGA GAT CAG TCC TTC GGT CTC GTG GGC TCG GAG
ATG TGT ATA AGA GAC AG
SEQ ID 83:
CAA GCA GAA GAC GGC ATA CGA GAT TGA CGG TAG AGT CTC GTG GGC TCG GAG
ATG TGT ATA AGA GAC AG
SEQ ID 84:
CAA GCA GAA GAC GGC ATA CGA GAT CGA TAT TAC CGT CTC GTG GGC TCG GAG
ATG TGT ATA AGA GAC AG
SEQ ID 85:
CAA GCA GAA GAC GGC ATA CGA GAT AAC TCG CAC AGT CTC GTG GGC TCG GAG
ATG TGT ATA AGA GAC AG
SEQ ID 86:
CAA GCA GAA GAC GGC ATA CGA GAT TCA GCG ATA AGT CTC GTG GGC TCG GAG
ATG TGT ATA AGA GAC AG
SEQ ID 87:
CAA GCA GAA GAC GGC ATA CGA GAT GAA CTC AAC CGT CTC GTG GGC TCG GAG
ATG TGT ATA AGA GAC AG
SEQ ID 88:
CAA GCA GAA GAC GGC ATA CGA GAT GGC GTC CAA TGT CTC GTG GGC TCG GAG
ATG TGT ATA AGA GAC AG
SEQ ID 89:
CAA GCA GAA GAC GGC ATA CGA GAT CAC ACA CAT AGT CTC GTG GGC TCG GAG
ATG TGT ATA AGA GAC AG
SEQ ID 90:
CAA GCA GAA GAC GGC ATA CGA GAT GCG ATA ACT AGT CTC GTG GGC TCG GAG
ATG TGT ATA AGA GAC AG
SEQ ID 91:
CAA GCA GAA GAC GGC ATA CGA GAT ATC ACC TGC AGT CTC GTG GGC TCG GAG
ATG TGT ATA AGA GAC AG
SEQ ID 92:
CAA GCA GAA GAC GGC ATA CGA GAT TAG CTT CAC AGT CTC GTG GGC TCG GAG
ATG TGT ATA AGA GAC AG
SEQ ID 93:
CAA GCA GAA GAC GGC ATA CGA GAT TCA GGA GTT GGT CTC GTG GGC TCG GAG
ATG TGT ATA AGA GAC AG
SEQ ID 94:
CAA GCA GAA GAC GGC ATA CGA GAT CGT TGC TAC AGT CTC GTG GGC TCG GAG
ATG TGT ATA AGA GAC AG
SEQ ID 95:
CAA GCA GAA GAC GGC ATA CGA GAT TGC GTT ACC GGT CTC GTG GGC TCG GAG
ATG TGT ATA AGA GAC AG
SEQ ID 96:
CAA GCA GAA GAC GGC ATA CGA GAT CTG CTG CTA CGT CTC GTG GGC TCG GAG
ATG TGT ATA AGA GAC AG
SEQ ID 97:
CAA GCA GAA GAC GGC ATA CGA GAT TCG GCT GTA GGT CTC GTG GGC TCG GAG
ATG TGT ATA AGA GAC AG
SEQ ID 98:
AAT GAT ACG GCG ACC ACC GAG ATC TAC ACC TGA TTA GGA TCG TCG GCA GCG
TCA GAT GTG TAT AAG AGA CAG

SEQ ID NOs 10-98 are adapter oligonucleotides. SEQ ID NOs 10-97 were used to generate the i7 Synaptic complexes 1, while SEQ ID NO 98 was used to generate the i5 Synaptic complex 2.

SEQ ID 99: ME′ (5′-phosphorylated complement of the transposase mosaic end sequence):
/5Phos/CTG TCT CTT ATA CAC ATC T/3InvdT/
SEQ ID 100: Universal primer 1:
CAA GCA GAA GAC GGC ATA CGA G
SEQ ID 101: Universal primer 2:
AAT GAT ACG GCG ACC ACC GAG

After thermal cycling, 10 μL of each amplified plasmid library resulting from the one-step reactions were pooled and purified with 0.75 volumetric equivalents of MAGwise™ paramagnetic beads according to the manufacturer's instructions (seqWell, Inc.). The pooled, purified libraries were sequenced on a MiSeq® sequencer (ILLUMINA®), and the reads were then demultiplexed based on the 10 base i7 indexes encoded in SEQ ID 10-SEQ ID 97. If additional multiplexing is needed, additional adapter oligo nucleotides containing i5 can be used to generate additional Synaptic complexes 2. Sequencing reads generated therefrom can then be demultiplexed based on both the i7 and i5 indexes encoded in the adapter oligonucleotides used to generate Synaptic complexes 1 and Synaptic complexes 2.

### Results

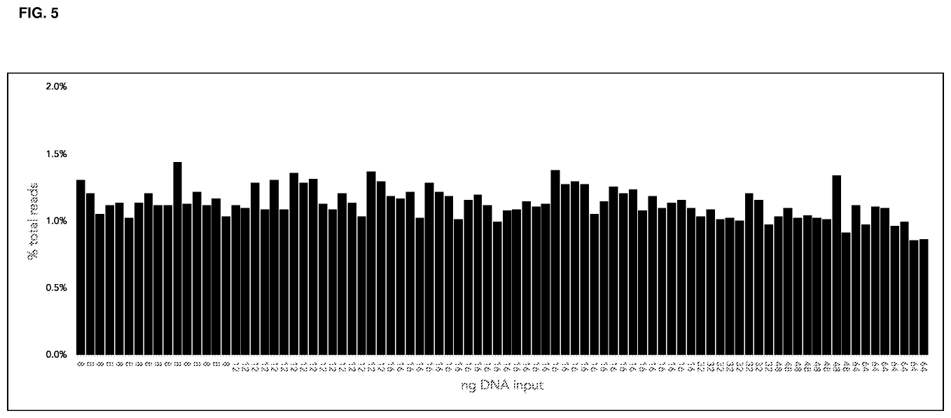

The read output balance for the plasmid samples over the 8-64 ng input range had a coefficient of variation (c.v.) of 10.2%. The read balance is shown in FIG. 5 and the normalization results are shown in Table 5.

TABLE 5
Normalization results (input vs. output)

| | Input (ng DNA) | Output Reads |
|---|---|---|
| Max | 64 | 1.4% |
| Min | 8 | 0.9% |
| Range (max/min) | 8 | 1.68 |

### OTHER EMBODIMENTS

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Other embodiments are in the claims.

1. A kit comprising:
(a) a first composition comprising a DNA polymerase;
(b) a second composition comprising:
(i) a first synaptic complex comprising a first transposase and a first adapter oligonucleotide; and
(ii) a second synaptic complex comprising a second transposase and a second adapter oligonucleotide;
wherein the second composition does not comprise magnesium ions; and
(c) magnesium ions, wherein the magnesium ions are either in a third composition or in the first composition.

2. The kit of claim 1, wherein:
the first adapter oligonucleotide comprises a first universal primer region and a first adapter barcode region; and
the second adapter oligonucleotide comprises a second universal primer region and a second adapter barcode region.

3. The kit of claim 1, wherein the first adapter oligonucleotide comprises a first adapter priming region and the second adapter oligonucleotide comprises a second adapter priming region.

4. The kit of claim 3, further comprising:
(i) a first amplifier oligonucleotide comprising a first universal primer region, a first amplifier barcode region, and a first amplifier priming region; and
(ii) a second amplifier oligonucleotide comprising a second universal primer region, a second amplifier barcode region, and a second amplifier priming region,
wherein the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

5. A method of generating a library from a nucleic acid sample comprising a target nucleic acid in a single-pot reaction in a first reaction vessel, wherein the method comprises amplifying the nucleic acid sample using the kit of claim 2 to generate sequencing oligonucleotides comprising:
(a) a nucleic acid sequence comprising the first universal primer region, the first adapter barcode region, a homologous sequence of a first nucleic acid fragment, the complement sequence of the second adapter barcode region, and the complement sequence of the second universal primer region, wherein a nucleic acid duplex comprising the first nucleic acid fragment and its complement is generated from the nucleic acid sample by transposase activity; and
(b) the complement sequence thereof,
thereby generating the library.

6. The method of claim 5, further comprising:
(i) combining the first composition, the second composition, magnesium ions, and the nucleic acid sample in the first reaction vessel;
(ii) generating intermediate nucleic acids comprising nucleic acid sequences of
(a) the first universal primer region, the first adapter barcode region, and the homologous sequence of the first nucleic acid fragment; and
(b) the second universal primer region, the second adapter barcode region, and the complement sequence of the first nucleic acid fragment,
in a transposition reaction between the nucleic acid sample, the first synaptic complex, and the second synaptic complex; and
(iii) generating the sequencing oligonucleotides in a polymerization reaction involving the intermediate nucleic acids and the DNA polymerase, wherein the polymerization reaction extends the 3′ ends of a nucleic acid duplex comprising a pair of the intermediate nucleic acids to generate the sequencing oligonucleotides.

7. The method of claim 6, wherein the transposition reaction occurs at a transposition reaction temperature between 25-65° C. and/or the polymerization reaction occurs at a polymerization reaction temperature between 55-95° C.

8. The method of claim 6, wherein the transposition reaction occurs for a first reaction duration between 1 and 30 minutes, and/or the polymerization reaction occurs for a second reaction duration between 1 and 60 minutes.

9. The method of claim 6, wherein the nucleic acid sample, the first adapter oligonucleotide, the second adapter oligonucleotide, the pair of intermediate nucleic acids, and/or the sequencing oligonucleotides comprise DNA.

10. The method of claim 9, wherein the nucleic acid sample comprises double-stranded DNA (dsDNA).

11. The method of claim 6, further comprising amplifying the library in the first reaction vessel in a PCR reaction with a first universal primer and a second universal primer, and wherein the first universal primer comprises a sequence homologous to the first universal primer region and the second universal primer comprises a sequence homologous to the second universal primer region, thereby generating an amplified library.

12. The method of claim 11, wherein the library, the first universal primer, and the second universal primer comprise DNA.

13. A method of generating a library from a nucleic acid sample in a single-pot reaction in a first reaction vessel, wherein the method comprises amplifying the nucleic acid sample using the kit of claim 4 to generate amplicons comprising:
(a) a nucleic acid sequence comprising the first universal primer region, the first amplifier barcode region, the first amplifier priming region, a homologous sequence of a first nucleic acid fragment, the complement sequence of the second amplifier priming region, the complement sequence of the second amplifier barcode region, and the complement sequence of the second universal primer region, wherein a nucleic acid duplex comprising the first nucleic acid fragment and its complement is generated from the nucleic acid sample by transposase activity; and
(b) the complement sequence thereof,
thereby generating the library.

14. The method of claim 13, further comprising:
(i) combining the first composition, the second composition, magnesium ions, the first amplifier oligonucleotide, the second amplifier oligonucleotide, and the nucleic acid sample in the first reaction vessel;
(ii) generating intermediate nucleic acids comprising nucleic acid sequences of
(a) the first adapter priming region and the homologous sequence of the first nucleic acid fragment; and
(b) the second adapter priming region and the complement sequence of the first nucleic acid fragment,
in a transposition reaction between the nucleic acid sample, the first synaptic complex, and the second synaptic complex; and
(iii) generating the amplicons in a PCR reaction with a pair of the intermediate nucleic acids, DNA polymerase, the first amplifier oligonucleotide, and the second amplifier oligonucleotide.

15. The method of claim 14, wherein the transposition reaction occurs at a transposition reaction temperature between 25-65° C.

16. The method of claim 14, wherein the transposition reaction occurs for a first reaction duration between 5 and 30 minutes.

17. The method of claim 14, wherein the PCR reaction comprises 1-35 cycles.

18. The method of claim 14, wherein the nucleic acid sample, the first adapter oligonucleotide, the second adapter oligonucleotide, the first amplifier oligonucleotide, the second amplifier oligonucleotide, the intermediate nucleic acids, and/or the amplicons comprise DNA.

19. The method of claim 18, wherein the nucleic acid sample comprises double-stranded DNA (dsDNA).

20. The method of claim 14, further comprising amplifying the library in the first reaction vessel in a PCR reaction with a first universal primer and a second universal primer, and wherein the first universal primer comprises a sequence homologous to the first universal primer region and the second universal primer comprises a sequence homologous to the second universal primer region, thereby generating an amplified library.

21. The method of claim 20, wherein the library, the first universal primer, and the second universal primer comprise DNA.

22. A method of generating a library comprising amplicons from a nucleic acid sample in a single-pot reaction in a first reaction vessel comprising:
I. combining in the first reaction vessel:
(a) magnesium ions;
(b) a DNA polymerase;
(c) a first synaptic complex comprising a first transposase and a first adapter oligonucleotide comprising a first adapter priming region;
(d) a second synaptic complex comprising a second transposase and a second adapter oligonucleotide comprising a second adapter priming region;
(e) a first amplifier oligonucleotide comprising a first universal primer region, a first amplifier barcode region, and a first amplifier priming region; and
(f) a second amplifier oligonucleotide comprising a second universal primer region, a second amplifier barcode region, and a second amplifier priming region,
wherein the first adapter priming region is homologous to the first amplifier priming region, and the second adapter priming region is homologous to the second amplifier priming region;
II. generating intermediate nucleic acids comprising nucleic acid sequences of
(a) the first adapter priming region and a homologous sequence of a first nucleic acid fragment; and
(b) the second adapter priming region and a complement sequence of the first nucleic acid fragment,
in a transposition reaction between the nucleic acid sample, the first synaptic complex, and the second synaptic complex, wherein a nucleic acid duplex comprising the first nucleic acid fragment and its complement is generated from the nucleic acid sample by transposase activity; and
III. generating the amplicons in a PCR reaction involving a pair of the intermediate nucleic acids, the DNA polymerase, the first amplifier oligonucleotide, and the second amplifier oligonucleotide,
wherein the amplicons comprise:
(a) a nucleic acid sequence comprising the universal primer region, the first amplifier barcode region, the first amplifier priming region, a homologous sequence of the first nucleic acid fragment, the complement sequence of the second amplifier priming region, the complement sequence of the second amplifier barcode region, and the complement sequence of the second universal primer region; and
(b) the complement sequence thereof.

23. The method of claim 22, wherein the transposition reaction occurs at a transposition reaction temperature between 25-65° C.

24. The method of claim 22, wherein transposition reaction occurs for a first reaction duration between 5 and 30 minutes.

25. The method of claim 22, wherein the PCR reaction comprises 1-35 cycles.

26. The method of claim 22, wherein the nucleic acid sample, the first adapter oligonucleotide, the second adapter oligonucleotide, the first amplifier oligonucleotide, and the second amplifier oligonucleotide, the pair of intermediate nucleic acids, and the amplicons comprise DNA.

27. The method of claim 26, wherein the nucleic acid sample comprises double-stranded DNA (dsDNA).

28. The method of claim 22, further comprising amplifying the library in the first reaction vessel in a PCR reaction with a first universal primer and a second universal primer, and wherein the first universal primer comprises a sequence homologous to the first universal primer region and the second universal primer comprises a sequence homologous to the second universal primer region, thereby generating an amplified library.

29. The method of claim 28, wherein the library, the first universal primer, and the second universal primer comprise DNA.

30. A method of generating a library comprising sequencing oligonucleotides from a nucleic acid sample in a single-pot reaction in a first reaction vessel comprising:
I. combining in the first reaction vessel:
(a) magnesium ions;
(b) a DNA polymerase;
(c) a first synaptic complex comprising a first transposase and a first adapter oligonucleotide comprising a first universal primer region and a first adapter barcode region; and
(d) a second synaptic complex comprising a second transposase and a second adapter oligonucleotide comprising a second universal primer region and a second adapter barcode region;
II. generating intermediate nucleic acids comprising nucleic acid sequences of
(a) the first universal primer region, the first adapter barcode region, and a homologous sequence of a first nucleic acid fragment; and
(b) the second universal primer region, the second adapter barcode region, and a complement sequence of the first nucleic acid fragment,
in a transposition reaction between the nucleic acid sample, the first synaptic complex, and the second synaptic complex, wherein a nucleic acid duplex comprising the first nucleic acid fragment and its complement is generated from the nucleic acid sample by transposase activity; and
III. generating the sequencing oligonucleotides in a polymerization reaction involving a pair of the intermediate nucleic acids and the DNA polymerase, wherein the polymerization reaction extends the 3′ ends of a nucleic acid duplex comprising the pair of intermediate nucleic acids to generate the sequencing oligonucleotides,
wherein the sequencing oligonucleotides comprise:
(a) a nucleic acid sequence comprising the first universal primer region, the first adapter barcode region, the homologous sequence of a first nucleic acid fragment, the complement sequence of the second adapter barcode region, and the complement sequence of the second universal primer region; and
(b) the complement sequence thereof.

31. The method of claim 30, wherein transposition reaction occurs at a transposition reaction temperature between 25-65° C. and/or the polymerization reaction occurs at a polymerization reaction temperature between 55-95° C.

32. The method of claim 30, wherein the transposition reaction occurs for a first reaction duration between 1 and 30 minutes and/or the polymerization reaction occurs for a second reaction duration between 1 and 60 minutes.

33. The method of claim 30, wherein the nucleic acid sample, the first adapter oligonucleotide, the second adapter oligonucleotide, the pair of intermediate nucleic acids, and the sequencing oligonucleotides comprise DNA.

34. The method of claim 33, wherein the nucleic acid sample comprises double-stranded DNA (dsDNA).

35. The kit of claim 3, further comprising:
(i) a first amplifier oligonucleotide comprising a first universal primer region and a first amplifier priming region; and
(ii) a second amplifier oligonucleotide comprising a second universal primer region and a second amplifier priming region,
wherein the first adapter priming region of the first adapter oligonucleotide is homologous to the first amplifier priming region of the first amplifier oligonucleotide and the second adapter priming region of the second adapter oligonucleotide is homologous to the second amplifier priming region of the second amplifier oligonucleotide.

36. A method of generating a library from a nucleic acid sample in a single-pot reaction in a first reaction vessel, wherein the method comprises amplifying the nucleic acid sample using the kit of claim 35 to generate amplicons comprising:
(a) a nucleic acid sequence comprising the first universal primer region, the first amplifier priming region, a homologous sequence of a first nucleic acid fragment, the complement sequence of the second amplifier priming region, and the complement sequence of the second universal primer region, wherein a nucleic acid duplex comprising the first nucleic acid fragment and its complement is generated from the nucleic acid sample by transposase activity; and
(b) the complement sequence thereof,
thereby generating the library.

37. The method of claim 36, further comprising:
(i) combining the first composition, the second composition, magnesium ions, the first amplifier oligonucleotide, the second amplifier oligonucleotide, and the nucleic acid sample in the first reaction vessel;
(ii) generating intermediate nucleic acids comprising nucleic acid sequences of
(a) the first adapter priming region and the homologous sequence of the first nucleic acid fragment; and
(b) the second adapter priming region and the complement sequence of the first nucleic acid fragment,
in a transposition reaction between the nucleic acid sample, the first synaptic complex, and the second synaptic complex; and
(iii) generating the amplicons in a PCR reaction with a pair of the intermediate nucleic acids, DNA polymerase, the first amplifier oligonucleotide, and the second amplifier oligonucleotide.

38. The method of claim 37, wherein the transposition reaction occurs at a transposition reaction temperature between 25-65° C.

39. The method of claim 37, wherein the transposition reaction occurs for a first reaction duration between 5 and 30 minutes.

40. The method of claim 37, wherein the PCR reaction comprises 1-35 cycles.

41. The method of claim 37, wherein the nucleic acid sample, the first adapter oligonucleotide, the second adapter oligonucleotide, the first amplifier oligonucleotide, the second amplifier oligonucleotide, the intermediate nucleic acids, and/or the amplicons comprise DNA.

42. The method of claim 41, wherein the nucleic acid sample comprises double-stranded DNA (dsDNA).

43. The method of claim 37, further comprising amplifying the library in the first reaction vessel in a PCR reaction with a first universal primer and a second universal primer, and wherein the first universal primer comprises a sequence homologous to the first universal primer region and the second universal primer comprises a sequence homologous to the second universal primer region, thereby generating an amplified library.

44. The method of claim 43, wherein the library, the first universal primer, and the second universal primer comprise DNA.

45. A method of generating a library comprising amplicons from a nucleic acid sample in a single-pot reaction in a first reaction vessel comprising:
I. combining in the first reaction vessel:
(a) magnesium ions;
(b) a DNA polymerase;
(c) a first synaptic complex comprising a first transposase and a first adapter oligonucleotide comprising a first adapter priming region;
(d) a second synaptic complex comprising a second transposase and a second adapter oligonucleotide comprising a second adapter priming region;
(e) a first amplifier oligonucleotide comprising a first universal primer region, and a first amplifier priming region; and
(f) a second amplifier oligonucleotide comprising a second universal primer region, and a second amplifier priming region,
wherein the first adapter priming region is homologous to the first amplifier priming region, and the second adapter priming region is homologous to the second amplifier priming region;
II. generating intermediate nucleic acids comprising nucleic acid sequences of
(a) the first adapter priming region and a homologous sequence of a first nucleic acid fragment; and
(b) the second adapter priming region and a complement sequence of the first nucleic acid fragment,
in a transposition reaction between the nucleic acid sample, the first synaptic complex, and the second synaptic complex, wherein a nucleic acid duplex comprising the first nucleic acid fragment and its complement is generated from the nucleic acid sample by transposase activity; and
III. generating the amplicons in a PCR reaction involving a pair of the intermediate nucleic acids, the DNA polymerase, the first amplifier oligonucleotide, and the second amplifier oligonucleotide,
wherein the amplicons comprise:
(a) a nucleic acid sequence comprising the universal primer region, the first amplifier priming region, a homologous sequence of the first nucleic acid fragment, the complement sequence of the second amplifier priming region, and the complement sequence of the second universal primer region; and
(b) the complement sequence thereof.

46. The method of claim 45, wherein the transposition reaction occurs at a transposition reaction temperature between 25-65° C.

47. The method of claim 45, wherein transposition reaction occurs for a first reaction duration between 5 and 30 minutes.

48. The method of claim 45, wherein the PCR reaction comprises 1-35 cycles.

49. The method of claim 45, wherein the nucleic acid sample, the first adapter oligonucleotide, the second adapter oligonucleotide, the first amplifier oligonucleotide, and the second amplifier oligonucleotide, the pair of intermediate nucleic acids, and the amplicons comprise DNA.

50. The method of claim 49, wherein the nucleic acid sample comprises double-stranded DNA (dsDNA).

51. The method of claim 45, further comprising amplifying the library in the first reaction vessel in a PCR reaction with a first universal primer and a second universal primer, and wherein the first universal primer comprises a sequence homologous to the first universal primer region and the second universal primer comprises a sequence homologous to the second universal primer region, thereby generating an amplified library.


SEQUENCE LISTING (101 sequences)

SEQ ID NO: 1 - DNA, 66 bp, synthetic construct
  source 1..66: mol_type = other DNA
caagcagaagacggcatacgagatagtcaccagtctcgtgggctcggagatgtgtataagagacag

SEQ ID NO: 2 - DNA, 70 bp, synthetic construct
  source 1..70: mol_type = other DNA
aatgatacggcgaccaccgagatctacacaaggagtatcgtcggcagcgtcagatgtgtataagagacag

SEQ ID NO: 3 - DNA, 19 bp, synthetic construct
  source 1..19: mol_type = other DNA
ctgtctcttatacacatct

SEQ ID NO: 4 - DNA, 22 bp, synthetic construct
  source 1..22: mol_type = other DNA
caagcagaagacggcatacgag

SEQ ID NO: 5 - DNA, 21 bp, synthetic construct
  source 1..21: mol_type = other DNA
aatgatacggcgaccaccgag

SEQ ID NO: 6 - DNA, 49 bp, synthetic construct
  source 1..49: mol_type = other DNA
caagcagaagacggcatacgagatggaagagatagtctcgtgggctcgg

SEQ ID NO: 7 - DNA, 53 bp, synthetic construct
  source 1..53: mol_type = other DNA
aatgatacggcgaccaccgagatctacacccacaacttatcgtcggcagcgtc

SEQ ID NO: 8 - DNA, 33 bp, synthetic construct
  source 1..33: mol_type = other DNA
tcgtcggcagcgtcagatgtgtataagagacag

SEQ ID NO: 9 - DNA, 34 bp, synthetic construct
  source 1..34: mol_type = other DNA
gtctcgtgggctcggagatgtgtataagagacag

SEQ ID NO: 10 - DNA, 68 bp, synthetic construct
  source 1..68: mol_type = other DNA
caagcagaagacggcatacgagatgagttagttggtctcgtgggctcggagatgtgtataagagacag

SEQ ID NO: 11 - DNA, 68 bp, synthetic construct
  source 1..68: mol_type = other DNA
caagcagaagacggcatacgagataatccagtacgtctcgtgggctcggagatgtgtataagagacag

SEQ ID NO: 12 - DNA, 68 bp, synthetic construct
  source 1..68: mol_type = other DNA
caagcagaagacggcatacgagattcctccgatggtctcgtgggctcggagatgtgtataagagacag

SEQ ID NO: 13 - DNA, 68 bp, synthetic construct
  source 1..68: mol_type = other DNA
caagcagaagacggcatacgagatccatcagaatgtctcgtgggctcggagatgtgtataagagacag

SEQ ID NO: 14 - DNA, 68 bp, synthetic construct
  source 1..68: mol_type = other DNA
caagcagaagacggcatacgagatggtaacgatagtctcgtgggctcggagatgtgtataagagacag

SEQ ID NO: 15 - DNA, 68 bp, synthetic construct
  source 1..68: mol_type = other DNA
caagcagaagacggcatacgagattaacagaccagtctcgtgggctcggagatgtgtataagagacag

SEQ ID NO: 16 - DNA, 68 bp, synthetic construct
  source 1..68: mol_type = other DNA
caagcagaagacggcatacgagatcatgttggcagtctcgtgggctcggagatgtgtataagagacag

SEQ ID NO: 17 - DNA, 68 bp, synthetic construct
  source 1..68: mol_type = other DNA
caagcagaagacggcatacgagatcgtgtaatgagtctcgtgggctcggagatgtgtataagagacag

SEQ ID NO: 18 - DNA, 68 bp, synthetic construct
  source 1..68: mol_type = other DNA
caagcagaagacggcatacgagatgtcaggttatgtctcgtgggctcggagatgtgtataagagacag

SEQ ID NO: 19 - DNA, 68 bp, synthetic construct
  source 1..68: mol_type = other DNA
caagcagaagacggcatacgagattggaaccgcagtctcgtgggctcggagatgtgtataagagacag

SEQ ID NO: 20 - DNA, 68 bp, synthetic construct
  source 1..68: mol_type = other DNA
caagcagaagacggcatacgagatggcaagtattgtctcgtgggctcggagatgtgtataagagacag

SEQ ID NO: 21 - DNA, 68 bp, synthetic construct
  source 1..68: mol_type = other DNA
caagcagaagacggcatacgagatttccgcgattgtctcgtgggctcggagatgtgtataagagacag

SEQ ID NO: 22 - DNA, 68 bp, synthetic construct
  source 1..68: mol_type = other DNA
caagcagaagacggcatacgagattcgcgttctggtctcgtgggctcggagatgtgtataagagacag

SEQ ID NO: 23 - DNA, 68 bp, synthetic construct
  source 1..68: mol_type = other DNA
caagcagaagacggcatacgagatccttggttcagtctcgtgggctcggagatgtgtataagagacag

SEQ ID NO: 24 - DNA, 68 bp, synthetic construct
  source 1..68: mol_type = other DNA
caagcagaagacggcatacgagatctgactaacagtctcgtgggctcggagatgtgtataagagacag

SEQ ID NO: 25 - DNA, 68 bp, synthetic construct
  source 1..68: mol_type = other DNA
caagcagaagacggcatacgagatttacctactggtctcgtgggctcggagatgtgtataagagacag

SEQ ID NO: 26 - DNA, 68 bp, synthetic construct
  source 1..68: mol_type = other DNA
caagcagaagacggcatacgagatgaagtacctggtctcgtgggctcggagatgtgtataagagacag

SEQ ID NO: 27 - DNA, 68 bp, synthetic construct
  source 1..68: mol_type = other DNA
caagcagaagacggcatacgagatcatattcctggtctcgtgggctcggagatgtgtataagagacag

SEQ ID NO: 28 - DNA, 68 bp, synthetic construct
  source 1..68: mol_type = other DNA
caagcagaagacggcatacgagatattgtgcgttgtctcgtgggctcggagatgtgtataagagacag

SEQ ID NO: 29 - DNA, 68 bp, synthetic construct
  source 1..68: mol_type = other DNA
caagcagaagacggcatacgagatccttctcaatgtctcgtgggctcggagatgtgtataagagacag

SEQ ID NO: 30 - DNA, 68 bp, synthetic construct
  source 1..68: mol_type = other DNA
caagcagaagacggcatacgagataaggtgcaatgtctcgtgggctcggagatgtgtataagagacag

SEQ ID NO: 31 - DNA, 68 bp, synthetic construct
  source 1..68: mol_type = other DNA
caagcagaagacggcatacgagatgtatccacttgtctcgtgggctcggagatgtgtataagagacag

SEQ ID NO: 32 - DNA, 68 bp, synthetic construct
  source 1..68: mol_type = other DNA
caagcagaagacggcatacgagatgcatcttatcgtctcgtgggctcggagatgtgtataagagacag

SEQ ID NO: 33 - DNA, 68 bp, synthetic construct
  source 1..68: mol_type = other DNA
caagcagaagacggcatacgagattgtcgaggtagtctcgtgggctcggagatgtgtataagagacag

SEQ ID NO: 34 - DNA, 68 bp, synthetic construct
  source 1..68: mol_type = other DNA
caagcagaagacggcatacgagattggctcggttgtctcgtgggctcggagatgtgtataagagacag

SEQ ID NO: 35 - DNA, 68 bp, synthetic construct
  source 1..68: mol_type = other DNA
caagcagaagacggcatacgagatcttgaatccagtctcgtgggctcggagatgtgtataagagacag

SEQ ID NO: 36 - DNA, 68 bp, synthetic construct
  source 1..68: mol_type = other DNA
caagcagaagacggcatacgagatgtatctgttggtctcgtgggctcggagatgtgtataagagacag

SEQ ID NO: 37 - DNA, 68 bp, synthetic construct
  source 1..68: mol_type = other DNA
caagcagaagacggcatacgagatgagttaccttgtctcgtgggctcggagatgtgtataagagacag

SEQ ID NO: 38 - DNA, 68 bp, synthetic construct
  source 1..68: mol_type = other DNA
caagcagaagacggcatacgagatcgtattgtcagtctcgtgggctcggagatgtgtataagagacag

SEQ ID NO: 39 - DNA, 68 bp, synthetic construct
  source 1..68: mol_type = other DNA
caagcagaagacggcatacgagatatgcacaatcgtctcgtgggctcggagatgtgtataagagacag

SEQ ID NO: 40 - DNA, 68 bp, synthetic construct
  source 1..68: mol_type = other DNA
caagcagaagacggcatacgagataatggtgtgcgtctcgtgggctcggagatgtgtataagagacag

SEQ ID NO: 41 - DNA, 68 bp, synthetic construct
  source 1..68: mol_type = other DNA
caagcagaagacggcatacgagatggaataacgagtctcgtgggctcggagatgtgtataagagacag

SEQ ID NO: 42 - DNA, 68 bp, synthetic construct
  source 1..68: mol_type = other DNA
caagcagaagacggcatacgagataatgacgttggtctcgtgggctcggagatgtgtataagagacag

SEQ ID NO: 43 - DNA, 68 bp, synthetic construct
  source 1..68: mol_type = other DNA
caagcagaagacggcatacgagatcatcgttcttgtctcgtgggctcggagatgtgtataagagacag

SEQ ID NO: 44 - DNA, 68 bp, synthetic construct
  source 1..68: mol_type = other DNA
caagcagaagacggcatacgagatgtaattgcaggtctcgtgggctcggagatgtgtataagagacag

SEQ ID NO: 45 - DNA, 68 bp, synthetic construct
  source 1..68: mol_type = other DNA
caagcagaagacggcatacgagatatcatcggcggtctcgtgggctcggagatgtgtataagagacag

SEQ ID NO: 46 - DNA, 68 bp, synthetic construct
  source 1..68: mol_type = other DNA
caagcagaagacggcatacgagatgtcttgtccggtctcgtgggctcggagatgtgtataagagacag

SEQ ID NO: 47 - DNA, 68 bp, synthetic construct
  source 1..68: mol_type = other DNA
caagcagaagacggcatacgagatgaggagtacggtctcgtgggctcggagatgtgtataagagacag

SEQ ID NO: 48 - DNA, 68 bp, synthetic construct
  source 1..68: mol_type = other DNA
caagcagaagacggcatacgagataactcatgcagtctcgtgggctcggagatgtgtataagagacag

SEQ ID NO: 49 - DNA, 68 bp, synthetic construct
  source 1..68: mol_type = other DNA
caagcagaagacggcatacgagatgctccagattgtctcgtgggctcggagatgtgtataagagacag

SEQ ID NO: 50 - DNA, 68 bp, synthetic construct
  source 1..68: mol_type = other DNA
caagcagaagacggcatacgagattctgaccaatgtctcgtgggctcggagatgtgtataagagacag

SEQ ID NO: 51 - DNA, 68 bp, synthetic construct
  source 1..68: mol_type = other DNA
caagcagaagacggcatacgagataacaggtcttgtctcgtgggctcggagatgtgtataagagacag

SEQ ID NO: 52 - DNA, 68 bp, synthetic construct
  source 1..68: mol_type = other DNA
caagcagaagacggcatacgagatttcattagcggtctcgtgggctcggagatgtgtataagagacag

SEQ ID NO: 53 - DNA, 68 bp, synthetic construct
  source 1..68: mol_type = other DNA
caagcagaagacggcatacgagatatgcactctggtctcgtgggctcggagatgtgtataagagacag

SEQ ID NO: 54 - DNA, 68 bp, synthetic construct
  source 1..68: mol_type = other DNA
caagcagaagacggcatacgagattcagcttaccgtctcgtgggctcggagatgtgtataagagacag

SEQ ID NO: 55 - DNA, 68 bp, synthetic construct
  source 1..68: mol_type = other DNA
caagcagaagacggcatacgagatcactgaatacgtctcgtgggctcggagatgtgtataagagacag

SEQ ID NO: 56 - DNA, 68 bp, synthetic construct
  source 1..68: mol_type = other DNA
caagcagaagacggcatacgagataacgtagcgcgtctcgtgggctcggagatgtgtataagagacag

SEQ ID NO: 57 - DNA, 68 bp, synthetic construct
  source 1..68: mol_type = other DNA
caagcagaagacggcatacgagatgctatgacaagtctcgtgggctcggagatgtgtataagagacag

SEQ ID NO: 58 - DNA, 68 bp, synthetic construct
  source 1..68: mol_type = other DNA
caagcagaagacggcatacgagattccgcaacatgtctcgtgggctcggagatgtgtataagagacag

SEQ ID NO: 59 - DNA, 68 bp, synthetic construct
  source 1..68: mol_type = other DNA
caagcagaagacggcatacgagatggttagcattgtctcgtgggctcggagatgtgtataagagacag

SEQ ID NO: 60 - DNA, 68 bp, synthetic construct
  source 1..68: mol_type = other DNA
caagcagaagacggcatacgagattacgtgttacgtctcgtgggctcggagatgtgtataagagacag

SEQ ID NO: 61 - DNA, 68 bp, synthetic construct
  source 1..68: mol_type = other DNA
caagcagaagacggcatacgagattccgtatatcgtctcgtgggctcggagatgtgtataagagacag

SEQ ID NO: 62 - DNA, 68 bp, synthetic construct
  source 1..68: mol_type = other DNA
caagcagaagacggcatacgagatcctctcggatgtctcgtgggctcggagatgtgtataagagacag

SEQ ID NO: 63 - DNA, 68 bp, synthetic construct
  source 1..68: mol_type = other DNA
caagcagaagacggcatacgagatgtgtgaattggtctcgtgggctcggagatgtgtataagagacag

SEQ ID NO: 64 - DNA, 68 bp, synthetic construct
  source 1..68: mol_type = other DNA
caagcagaagacggcatacgagatgaactgacaagtctcgtgggctcggagatgtgtataagagacag

SEQ ID NO: 65 - DNA, 68 bp, synthetic construct
  source 1..68: mol_type = other DNA
caagcagaagacggcatacgagataatggcgtatgtctcgtgggctcggagatgtgtataagagacag

SEQ ID NO: 66 - DNA, 68 bp, synthetic construct
  source 1..68: mol_type = other DNA
caagcagaagacggcatacgagatgttattggtggtctcgtgggctcggagatgtgtataagagacag

SEQ ID NO: 67 - DNA, 68 bp, synthetic construct
  source 1..68: mol_type = other DNA
caagcagaagacggcatacgagatttgtggacgcgtctcgtgggctcggagatgtgtataagagacag

SEQ ID NO: 68 - DNA, 68 bp, synthetic construct
  source 1..68: mol_type = other DNA
caagcagaagacggcatacgagataagcgcacatgtctcgtgggctcggagatgtgtataagagacag

SEQ ID NO: 69 - DNA, 68 bp, synthetic construct
  source 1..68: mol_type = other DNA
caagcagaagacggcatacgagatgcactagatagtctcgtgggctcggagatgtgtataagagacag

SEQ ID NO: 70 - DNA, 68 bp, synthetic construct
  source 1..68: mol_type = other DNA
caagcagaagacggcatacgagatcattgtaggcgtctcgtgggctcggagatgtgtataagagacag

SEQ ID NO: 71 - DNA, 68 bp, synthetic construct
  source 1..68: mol_type = other DNA
caagcagaagacggcatacgagataactgagcttgtctcgtgggctcggagatgtgtataagagacag

SEQ ID NO: 72 - DNA, 68 bp, synthetic construct
  source 1..68: mol_type = other DNA
caagcagaagacggcatacgagattctaagcgtagtctcgtgggctcggagatgtgtataagagacag

SEQ ID NO: 73 - DNA, 68 bp, synthetic construct
  source 1..68: mol_type = other DNA
caagcagaagacggcatacgagatcctagtggatgtctcgtgggctcggagatgtgtataagagacag

SEQ ID NO: 74 - DNA, 68 bp, synthetic construct
  source 1..68: mol_type = other DNA
caagcagaagacggcatacgagataagacggacagtctcgtgggctcggagatgtgtataagagacag

SEQ ID NO: 75 - DNA, 68 bp, synthetic construct
  source 1..68: mol_type = other DNA
caagcagaagacggcatacgagatgttactcatggtctcgtgggctcggagatgtgtataagagacag

SEQ ID NO: 76 - DNA, 68 bp, synthetic construct
  source 1..68: mol_type = other DNA
caagcagaagacggcatacgagatattcgcgcaggtctcgtgggctcggagatgtgtataagagacag

SEQ ID NO: 77 - DNA, 68 bp, synthetic construct
  source 1..68: mol_type = other DNA
caagcagaagacggcatacgagatcatactacaggtctcgtgggctcggagatgtgtataagagacag

SEQ ID NO: 78 - DNA, 68 bp, synthetic construct
  source 1..68: mol_type = other DNA
caagcagaagacggcatacgagattgtacggctcgtctcgtgggctcggagatgtgtataagagacag

SEQ ID NO: 79 - DNA, 68 bp, synthetic construct
  source 1..68: mol_type = other DNA
caagcagaagacggcatacgagatcctaagaatggtctcgtgggctcggagatgtgtataagagacag

SEQ ID NO: 80 - DNA, 68 bp, synthetic construct
  source 1..68: mol_type = other DNA
caagcagaagacggcatacgagatttgtcggtcagtctcgtgggctcggagatgtgtataagagacag

SEQ ID NO: 81 - DNA, 68 bp, synthetic construct
  source 1..68: mol_type = other DNA
caagcagaagacggcatacgagatcggttacacagtctcgtgggctcggagatgtgtataagagacag

SEQ ID NO: 82 - DNA, 68 bp, synthetic construct
  source 1..68: mol_type = other DNA
caagcagaagacggcatacgagatcagtccttcggtctcgtgggctcggagatgtgtataagagacag

SEQ ID NO: 83 - DNA, 68 bp, synthetic construct
  source 1..68: mol_type = other DNA
caagcagaagacggcatacgagattgacggtagagtctcgtgggctcggagatgtgtataagagacag

SEQ ID NO: 84 - DNA, 68 bp, synthetic construct
  source 1..68: mol_type = other DNA
caagcagaagacggcatacgagatcgatattaccgtctcgtgggctcggagatgtgtataagagacag

SEQ ID NO: 85 - DNA, 68 bp, synthetic construct
  source 1..68: mol_type = other DNA
caagcagaagacggcatacgagataactcgcacagtctcgtgggctcggagatgtgtataagagacag

SEQ ID NO: 86 - DNA, 68 bp, synthetic construct
  source 1..68: mol_type = other DNA
caagcagaagacggcatacgagattcagcgataagtctcgtgggctcggagatgtgtataagagacag

SEQ ID NO: 87 - DNA, 68 bp, synthetic construct
  source 1..68: mol_type = other DNA
caagcagaagacggcatacgagatgaactcaaccgtctcgtgggctcggagatgtgtataagagacag

SEQ ID NO: 88 - DNA, 68 bp, synthetic construct
  source 1..68: mol_type = other DNA
caagcagaagacggcatacgagatggcgtccaatgtctcgtgggctcggagatgtgtataagagacag

SEQ ID NO: 89 - DNA, 68 bp, synthetic construct
  source 1..68: mol_type = other DNA
caagcagaagacggcatacgagatcacacacatagtctcgtgggctcggagatgtgtataagagacag

SEQ ID NO: 90 - DNA, 68 bp, synthetic construct
  source 1..68: mol_type = other DNA
caagcagaagacggcatacgagatgcgataactagtctcgtgggctcggagatgtgtataagagacag

SEQ ID NO: 91 - DNA, 68 bp, synthetic construct
  source 1..68: mol_type = other DNA
caagcagaagacggcatacgagatatcacctgcagtctcgtgggctcggagatgtgtataagagacag

SEQ ID NO: 92 - DNA, 68 bp, synthetic construct
  source 1..68: mol_type = other DNA
caagcagaagacggcatacgagattagcttcacagtctcgtgggctcggagatgtgtataagagacag

SEQ ID NO: 93 - DNA, 68 bp, synthetic construct
  source 1..68: mol_type = other DNA
caagcagaagacggcatacgagattcaggagttggtctcgtgggctcggagatgtgtataagagacag

SEQ ID NO: 94 - DNA, 68 bp, synthetic construct
  source 1..68: mol_type = other DNA
caagcagaagacggcatacgagatcgttgctacagtctcgtgggctcggagatgtgtataagagacag

SEQ ID NO: 95 - DNA, 68 bp, synthetic construct
  source 1..68: mol_type = other DNA
caagcagaagacggcatacgagattgcgttaccggtctcgtgggctcggagatgtgtataagagacag

SEQ ID NO: 96 - DNA, 68 bp, synthetic construct
  source 1..68: mol_type = other DNA
caagcagaagacggcatacgagatctgctgctacgtctcgtgggctcggagatgtgtataagagacag

SEQ ID NO: 97 - DNA, 68 bp, synthetic construct
  source 1..68: mol_type = other DNA
caagcagaagacggcatacgagattcggctgtaggtctcgtgggctcggagatgtgtataagagacag

SEQ ID NO: 98 - DNA, 72 bp, synthetic construct
  source 1..72: mol_type = other DNA
aatgatacggcgaccaccgagatctacacctgattaggatcgtcggcagcgtcagatgtgtataagagacag

SEQ ID NO: 99 - DNA, 19 bp, synthetic construct
  source 1..19: mol_type = other DNA
ctgtctcttatacacatct

SEQ ID NO: 100 - DNA, 22 bp, synthetic construct
  source 1..22: mol_type = other DNA
caagcagaagacggcatacgag

SEQ ID NO: 101 - DNA, 21 bp, synthetic construct
  source 1..21: mol_type = other DNA
aatgatacggcgaccaccgag